(19) United States
(12) Patent Application Publication
(10) Pub. No.: US 2009/0221624 A1
(43) Pub. Date: Sep. 3, 2009

(54) 4-AMINOQUINOLINE COMPOUNDS FOR TREATING VIRUS-RELATED CONDITIONS

(76) Inventors: Paul D. Olivo, Clayton, MO (US); Benjamin A. Buscher, Madison, WI (US); Julie Dyall, Bethesda, MD (US); Jennifer I. Jocket-Balsarotti, St. Louis, MO (US); Andrew K. O'Guin, Waterloo, IL (US); Robert M. Roth, St. Louis, MO (US); Yi Zhou, Lexington, MA (US); Gary W. Franklin, Godfrey, IL (US); Gale W. Starkey, Richmond Heights, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/913,530

(22) PCT Filed: May 4, 2006
(86) PCT No.: PCT/US06/17200

§ 371 Date: Jul. 15, 2008

Related U.S. Application Data
(60) Provisional application No. 60/678,917, filed on May 6, 2005.

(51) Int. Cl. A61K 31/435; A61K 31/47; A61P 31/12

(52) U.S. Cl. 514/290; 514/313

(57) ABSTRACT

This invention is directed to aminoquinoline compounds, pharmaceutical compositions of such compounds, kits comprising such compounds, and uses of such compounds for preparing medicaments and treating virus-related conditions in animals.

### CROSS-REFERENCE TO RELATED APPLICATIONS

This application claims the benefit of priority of U.S. provisional application Ser. No. 60/678,917 filed May 6, 2005, and PCT application PCT/US2006/017200 filed May 4, 2006. These applications are incorporated herein by reference in their entireties.

### GOVERNMENT RIGHTS

This invention was developed at least in part with the support of Grant Number 2 R44 A1047552-02A1 from the National Institutes of Health. The government of the United States of America has certain rights in this work.

### INTRODUCTION

This invention is directed generally to 4-aminoquinoline compounds, combinations of such compounds, combinations with antiviral agents, and their use in treating virus-related conditions.

Significant progress has been made in the development of antiviral drugs. Just thirty years ago, there were no FDA-approved antiviral drugs. Today, there are over forty. Still, the applicability of these drugs continues to be limited. More than half the FDA-approved antiviral drugs, for example, are for use in treating infections caused by human immunodeficiency virus (HIV), and most of the remaining drugs are for use in treating herpes virus infections.

The majority of human viral pathogens are RNA viruses. The FDA has approved only two small-molecule antiviral drugs for use against RNA viruses, and only one, ribavirin, exhibits broad-spectrum activity. Consequently, for many RNA viruses, there are only limited, if any, therapeutic options available.

Hepatitis C virus (HCV), for example, is an RNA virus that causes chronic hepatitis afflicting an estimated 170 million individuals worldwide, including approximately 4 million in the United States. The level of sickness and mortality associated with HCV is high. The majority of infected subjects remain infected for life, and a significant percentage of HCV-infected subjects ultimately progress to cirrhosis, liver failure, or hepatocellular carcinoma. No vaccine has been reported for HCV. It is believed that the best available treatment for HCV is a combination of two broad-spectrum antiviral agents, interferon and ribavirin. This therapy, however, is only effective for about half of all subjects, and is associated with serious side effects that cause another 10-15% of otherwise suitable subjects to discontinue therapy. HCV also tends to develop resistance to antiviral drugs during therapy. It is expected, therefore, that multi-drug “cocktail” therapies, used in different combinations at varying stages of disease progression, will be necessary to effectively manage chronic HCV infection.

Respiratory syncytial virus (RSV) is another RNA virus. This virus is contracted by virtually all children by the age of three. It spreads rapidly through contact with respiratory secretions, and is the primary cause of bronchopneumonia in infants and children in the United States. It is estimated that RSV infections result in 100,000 hospitalizations and 4,000 deaths each year in the United States alone. Premature infants, immunodeficient subjects, and the institutionalized elderly are at the greatest risk for sickness and mortality from RSV. Current treatments for RSV are limited and suboptimal. For example, inhaled ribavirin is difficult to administer and relatively toxic, and, as a result, infrequently used. A prophylactically-administrated monoclonal antibody (Synagis, Medimmune) also is available, but used only with high-risk subjects.

Primary screening programs to identify compounds with antiviral activity involve two general methods—“targeted” screening and “unbiased” screening. In the “targeted” approach, a particular biochemical target is chosen, and then inhibitors of the chosen target are screened. The chosen target is typically an enzyme or a receptor that is known or thought to be essential to viral replication. In the alternative approach, “unbiased” screening, inhibitors of viral replication are sought without a priori concern for the target. Unbiased screening necessarily involves the use of cell culture for virus replication. This is due, in part, to the fact that cellular targets are likely targets of many antiviral agents. Although cell-based screening has been used successfully throughout the drug-discovery field, it has historically been problematic when screening for antiviral compounds because it required inoculation of an infectious virus onto the cells, and then producing additional infectious progeny virus. Handling such infectious material is not easily compatible with the high throughput process of screening large libraries of compounds.

Partial viral replication systems have been developed to circumvent the problems associated with cell-based cultures using whole viral systems. In the partial viral systems, viral genomes lacking one or more genetic elements essential for complete replication are used to accomplish viral genomic replication without producing the infectious virus. This is particularly important for viruses, such as hemorrhagic fever virus, classified as biohazard level 3 or 4 (BL-3 or BL4). A screening process that utilizes these incomplete viral genomes can identify inhibitors of any biochemical pathway involved in viral genome replication, transcription, and translation. This allows for screening with respect to multiple possible targets. These targets do not have to be known, thus making the screening process unbiased. In addition, the targets are pre-validated, given that inhibition of RNA replication is directly relevant to the viral disease process. Screening with partial viral replication systems additionally is advantageous because complex viral replication pathways can be easily monitored by measuring levels of viral RNA or expression of a reporter gene present in the replicon or defective genome.

The utility of using partial viral replication systems can be expanded further by screening for multiple viruses simultaneously. More specifically, by combining cell lines, each of which contain a partially replicating viral genome, one can screen for antiviral activity against each virus during the same screen, thereby saving time, reducing costs, and allowing for more effective use of material libraries. And, in addition to measuring the effect of a compound on genomic replication of several viruses, use of a partial viral replication system can provide information on the specificity of the antiviral effect. This information is helpful in accessing, for example, whether the effect is acting on a specific viral target or on a cellular target, and, thus, exerting its effect on the virus indirectly. This also may be helpful for identifying compounds that exhibit broad antiviral activity (i.e., activity against more than one, and typically several, viruses).

There are generally two types of partial viral replication systems: defective genomes and replicons.

Defective genomes (which often are artificial genomes or minigenomes) typically contain all the cis-acting elements required for viral genomic replication and transcription, but lack one or more of the genetic elements that encode the trans-acting factors required for replication. Such defective genomes, therefore, cannot replicate by themselves, but can replicate if the missing factor (or factors) is supplied in trans. When a cell contains both the defective genome and the necessary trans-acting factors, partial viral replication occurs within the cell without infectious virus being produced. Cell cultures containing replicating defective viral genomes represent a useful tool for antiviral drug discovery. For example, they may be used to observe the effect of an antiviral agent in the context of living cells, and therefore allow for the selection of agents that can enter and act within living cells. Such cell lines also may be used to immediately identify antiviral agents with undesirable cytotoxicity using well-established cytotoxicity assays. In addition, such cell lines permit cell-based drug discovery screens to be performed on a broad array of viruses, including, for example, viruses (e.g., HCV and Human Papillomavirus (HPV)) that are difficult to culture or cannot be cultured by conventional means. Further, such cell lines are much safer and thus easier to work with than cell lines that make infectious virus. A still further advantage of such cell lines is that reporter genes (e.g., luciferase, beta-galactosidase, secreted alkaline phosphatase, green fluorescent protein, etc.) that facilitate high throughput automated analysis of viral genome copy number can be incorporated into the defective genome.

Replicons are subgenomic nucleic acid molecules that are capable of replicating within cells cultured in vitro. In contrast to defective genomes, replicons typically encode all the cis and trans-acting viral components required for replication and transcription of the viral genome within a cell. Replicons, however, lack one or more elements required to replicate a full virus. For example, replicons often lack sequences related to infectivity. Such replicons are safer and easier to work with than a corresponding infectious virus, and are often ideal for studying treatments directed to viral replication because viral functions related to infectivity typically are not required for replication. Recently, several replicons capable of persistent replication in cells have been reported. See, e.g., Bartenschlager et al., “Novel cell culture systems for the hepatitis C virus,” Antiviral Res. 52:1-17 (2001). See also, Frolov et al., “Selection of RNA replicons capable of persistent noncytopathic replication in mammalian cells,” J. Virol. 73:3854-65 (1999). See also, Rice et al., “Transcription of infectious yellow fever RNA from full-length cDNA templates produced by in vitro ligation,” New Biol. 1:285-96 (1989). A viral replicon culture (VRC) is a cell line containing a persistently replicating non-cytopathic viral replicon. VRCs provide benefits similar to those described above with respect to the defective genome cell lines.

Partial viral replication systems are valuable molecular tools that can be used for a variety of purposes. In general, they can be used as tools to promote basic investigations of viral replication and pathogenesis. Specific uses include, for example:
a) identification and evaluation of antiviral agents (e.g., small molecules, RNAi, antisense, ribozymes, etc.),
b) development and evaluation of diagnostic assays and reagents,
c) development and evaluation of vaccines,
d) development of vectors (e.g., gene delivery, protein expression, vaccines, etc.),
e) evaluation of the function of viral genes and proteins,
f) localization of viral proteins,
g) characterization of cellular antiviral pathways (e.g., IFN, PKR^{X}, RNAi, etc.),
h) characterization of viral RNA replication (within cells and cell-free systems),
i) identification of cis-acting elements, and
j) identification of host proteins required for viral replication.

Screening with partial viral replication systems can be applied to any type of viral pathogen, including viruses with RNA or DNA genomes. Examples include those shown in Table 1:

TABLE 1
Viral Replicons for Antiviral Screening

| Family | Virus | Infectious clone? | Genome |
|---|---|---|---|
| Togaviridae | Sindbis | Yes | RNA (positive polarity) |
| | VEE | Yes | |
| | Semliki Forest virus | Yes | |
| | WEE | — | |
| | EEE | — | |
| | O'nyong Nyong | — | |
| | Ross river virus | Yes | |
| | Rubella virus | — | |
| Picornaviridae | Poliovirus | Yes | RNA (positive polarity) |
| | Coxsackievirus | Yes | |
| | Enterovirus | Yes | |
| | Hepatitis A | Yes | |
| Flaviviridae | Yellow fever | Yes | RNA (positive polarity) |
| | Dnegue 1-4 | Yes | |
| | West Nile | Yes | |
| | Japanese encephalitis | Yes | |
| | Hepatitis C | Yes | |
| | TBE | — | |
| | Kunjin | Yes | |
| | Omsk HF virus | — | |
| | Murray valley | — | |
| | Kyasanur | — | |
| | Rocio virus | — | |
| | BVDV | Yes | |
| Astroviridae | Astrovirus | Yes | RNA (positive polarity) |
| Caliciviridae | Norwalk | No | RNA (positive polarity) |
| | Murine calicivirus | — | |
| | feline calicivirus | Yes | |
| Coronaviridae | Human coronavirus | — | RNA (positive polarity) |
| | SARS | — | |
| Rhabdoviridae | Rabies virus | Yes | RNA (negative polarity) |
| | VSV | Yes | |
| Paramyxoviridae | RSV | Yes | RNA (negative polarity) |
| | Measles | Yes | |
| | Mumps | Yes | |
| | Human metapneumoviris | Yes | |
| Filoviridae | Ebola | Yes | RNA (negative polarity) |
| | Marburg | — | |
| Bunyaviridae | California encephalitis | Yes | RNA (negative polarity) |
| | Hanta virus | — | |
| | CCHF virus | — | |
| Arenaviridae | LCM | — | RNA (negative polarity) |
| | Lassa fever | — | |
| | Argentine HF | — | |
| | Bolivian HF | — | |
| Reoviridae | Colorado tick fever | — | RNA (double-stranded) |
| Hepadanviridae | Hepatitis B virus | Yes | DNA (partial duplex) |
| Papillomaviridae | Human papilloma virus | Yes | DNA (double-stranded) |
| Polyomaviridae | JC virus | Yes | DNA (double-stranded) |
| | BK virus | Yes | |
| Herpesviridae | Herpes simplex virus 1 | Yes | DNA (double-stranded) |
| | Herpes simplex virus 2 | Yes | |
| | Epstein Barr virus | Yes | |
| | Human cytomegalovirus | Yes | |
| | Varicella-zoster virus | Yes | |
| | Herpes simplex virus 6 | — | |
| | Herpes simplex virus 7 | — | |
| | Herpes simplex virus 8 | — | |
| Paravavoviridae | Human parvovirus | Yes | DNA (single-stranded) |

Many viruses in Table 1 are priority pathogens of concern for biodefense. One of these, viral hemorrhagic fever (VHF), refers to a group of illnesses that are caused by members of four families of viruses (Flaviviridae, Filoviridae, Arenaviridae, and Bunyaviridae). Many of these viruses cause severe, life-threatening diseases that are easily transmitted from person to person, and are listed by the CDC and NIH as category A pathogens. Category B and C viral pathogens include members of the Togaviridae (VEE, etc.), Flaviviridae (YFV, WNV, etc.) and Bunyaviridae families.

There have been significant advances in the development of reverse genetic systems for RNA viruses. Generally, all viruses possess genes that encode for RNA-dependent RNA polymerase to produce mRNA and thereby replicate its genome. Upon infection of cells, the genomic RNA of positive strand RNA viruses replicates through the use of a complementary negative strand intermediate in a three phase mechanism. In the first phase, translated viral proteins and one or more host proteins form a replicase complex that attaches to the 3′ of the positive-strand viral RNA. In the second phase, the viral RNA is copied to a complementary, negative-strand RNA. In the final phase, the negative RNA serves as a template for synthesis of progeny positive-strand viral RNA. Notably, positive-strand RNA viruses make structural proteins soon after entering the host cell cytoplasm because the viral genome acts as mRNA.

Positive-strand RNA viruses include, for example, members of the Togavilidae, Flaviviridae (e.g., HCV, WNV, and YFV), and Picornaviridae. These viruses were among the first RNA viruses for which reverse genetics studies were performed. See, e.g., Khromykh, “Replicon-based vectors of positive strand RNA viruses,” Curr. Opin. Mol. Ther. 2:555-69 (2000). See also, Khromykh et al., “Subgenomic replicons of the flavivirus Kunjin: construction and applications,” J. Virol. 71:1497-505 (1997). See also, Racaneillo, V. R., “Studying poliovirus with infectious cloned cDNA,” Rev. Infect. Dis. 6 Suppl 2:S514-5 (1984). See also, Rice et al., “Transcription of infectious yellow fever RNA from full-length cDNA templates produced by in vitro ligation,” New Biol. 1:285-96 (1989). There is, therefore, significant experience in genetically manipulating these viruses and constructing genetic tools such as cDNA clones (including infectious cDNA clones), subgenomic replicons, and defective genomes. See, e.g., Lai et al., “Infectious RNA transcribed from stably cloned full-length cDNA dengue type 4 virus,” Proc. Natl. Aced. Sci. USA 88:5139-43 (1991). See also, Perri el al., “Replicon vectors derived from Sindbis virus and Semliki forest virus that establish persistent replication in host cells,” J. Virol. 74:9802-7 (2000). See also, Sumiyoshi et al., “Infectious Japanese encephalitis virus RNA can be synthesized from in vitro-ligated cDNA templates,” J. Virol. 66:5425-31 (1992). In fact, the construction of infectious clones has become the standard practice for developing models to study these viruses. An infectious cDNA for yellow fever virus strain 17D, for example; was successfully constructed, and has been used in reverse genetics experiments to help elucidate the role of various viral genes in replication and pathogenesis. See, e.g., Rice et al., “Transcription of infectious yellow fever RNA from full-length cDNA templates produced by in vitro ligation,” New Biol. 1:285-96 (1989). This cDNA clone also has promoted efforts to identify the trans-acting and genomic cis-acting elements involved in viral replication. See Lindenbach, et al., “trans-Complementation of yellow fever virus NS1 reveals a role in early RNA replication,” J. Virol. 71:9608-17 (1997). Full-length cDNA also have enabled investigators to construct replicons. See, e.g., Lindenbach, et al.

Replicons and defective genomes can be used in cultures that lack cell toxicity, thus enabling long-term propagation and reporter or resistance gene expression. See Lindenbach, et al. Flavivirus replicons, for example, have been constructed by many groups, and these efforts have formed the foundation of the recent success in constructing subgenomic replicons of the hepatitis C virus. See, e.g., Blight et al., “Efficient initiation of HCV RNA replication in cell culture,” Science 290:1972-74 (2000). See also, Lohmann et al., “Replication of subgenomic hepatitis C virus RNAs in a hepatoma cell line,” Science 285: 110-3 (1999). Replicons also have been constructed for the closely related Kunjin flavivirus. See, e.g., Khromykh et al., J. Virol. 71:1497-505 (1997). A West Nile virus (WNV) replicon, modeled on Kunjin and HCV based on the New York 2000 strain of WNV (3000.0259), also has been constructed. See, e.g., Shi et al., “Construction and characterization of subgenomic replicons of New York strain of West Nile virus,” Virology 296:219-33 (2002) (57, 67). See also, Yamshchikov et al., “An infectious clone of the West Nile Flavivirus,” Virology 281:294-304 (2001).

The HCV genome, in particular, has been-analyzed extensively. See, e.g., Blight et al., “Molecular virology of hepatitis C virus: an update with respect to potential antiviral targets,” Antiviral Ther. 3:71-81 (1998). HCV has-been classified as a member of the family Flavividae, which includes flaviviruses (e.g., yellow fever virus) and pestiviruses (e.g., bovine viral diarrhea virus). A major impediment to understanding HCV virology has been the lack of a reliable and robust cell culture replication system. A second problem is that the only animal model with which to study. HCV pathogenesis is the chimpanzee. A full-length cDNA clone of the HCV genome has been constructed (consensus 1a). See Kolykhalov et al., “Transmission of hepatitis C by intrahepatic inoculation with transcribed RNA,” Science 277:570-4 (1997). In vitro transcripts made from this cDNA were shown to cause HCV hepatitis following intrahepatic injection into chimpanzees. Unfortunately, a robust cell culture system for propagating virus and making stocks of mutant viruses from an infectious cDNA clone is not yet available. Cell lines stably transformed with a subgenomic HCV RNA replicon derived from HCV RNA of genotype 1b have been isolated. See Frese et al., “Interferon-alpha inhibits hepatitis C virus subgenomic RNA replication by an MxA-independent pathway,” J. Gen. Virol. 82:723-33 (2001). See also, Lohmann et al., “Mutations in hepatitis C virus RNAs conferring cell culture adaptation,” J. Virol. 75:1437-49 (2001). Frese and Lohmann have reported evidence of HCV RNA replication occurring in the cytoplasm of these cells. Although not capable of generating hepatitis C virus, this system furthered efforts to demonstrate that bona fide HCV RNA replication can occur in a cell culture system. Recently cell lines containing an HCV replicon have reportedly been isolated. See Blight et al., Science 290:1972-74 (2000). These cells have been analyzed extensively and shown to contain an HCV replicon that exhibits autonomous HCV RNA replication. Over twenty cell lines that contained a constitutively replicating HCV replicon derived from genotype 1b were isolated. The entire replicon from five cell lines was sequenced, as were the NS5a open reading frame in an additional 17 clones. cDNA clones were derived from ten of the clones, which, in turn, were shown to contain adaptive mutations that conferred an increased efficiency of transfection (increased number of G418-resistant colonies following transfection). All these stable Huh7 replicon-containing cell lines, as well as the cDNA plasmids, can be used to generate new cell lines.

There are generally six families of negative-stranded RNA viruses that have human pathogenic members. Three are non-segmented (Paramyxoviridae (e.g., RSV), Rhabdomyxoviridae, and Filoviridae), and three are segmented (Orthomyxoviridae, Bunyaviridae, and Arenaviridae). In contrast to positive-strand RNA viruses, the RNA of negative-strand RNA viruses must first be copied into positive-strand mRNA before translation and viral replication can occur. In the first phase of a three phase mechanism, negative-strand RNA is transcribed in the host-cell to produce a positive-strand mRNA. In a second phase, the mRNA creates viral proteins including RNA-dependent RNA polymerase. In the last phase, new progeny negative-strand RNA are created.

Although replicons and defective genomes perse have not yet been described for negative-strand viruses, there have been significant advances in the genetic manipulation of various members of these virus families. See, e.g., Roberts et al., “Recovery of negative-stranded RNA viruses from plasmid DNAs: a positive approach revitalizes, a negative field,” Virology 247:1-6 (1998). Recovery of infectious rabies virus, Sendai virus, vesicular stomatitis virus, measles virus, and RSV, for example, has been accomplished using T7 RNA polymerase to generate full-length antigenomic transcripts from cDNA, together with the viral proteins necessary for nucleocapsid assembly and RNA replication and transcription. See, e.g., Garcin et al., “A highly recombinogenic system for the recovery of infectious Sendai paramyxovirus from cDNA: generation of a novel copy-back nondefective interfering virus,” Embo. J. 14:6087-94 (1995). See also, Radecke et al., “Rescue of measles viruses from cloned DNA,” Embo. J. 14:5773-84 (1995). See also, Schnell et al., “Infectious rabies viruses from cloned cDNA,” Embo. J. 13:4195-203 (1994). See also, Whelan et al., “Efficient recovery of infectious vesicular stomatitis virus entirely from cDNA clones,” Proc. Natl. Acad. Sci. USA 92:8388-92 (1995). An infectious cDNA clone for Ebola virus also has been developed. See, e.g., Neumann et al., “Reverse genetics demonstrates that proteolytic processing of Ebola virus glycoprotein is not essential for replication in cell culture,” J. Virol. 76:406-10 (2002).

Despite the foregoing advances, there are still very few small molecule broad-spectrum antiviral drugs. Consequently, there continues to be a critical medical need for effective therapies against viral pathogens. In addition, a need continues to exist for broad-spectrum antiviral therapies that are effective against both positive-strand and negative-strand RNA viruses.

### SUMMARY

Accordingly, the inventors herein have succeeded in devising a method for treating a viral infection in an animal. The method comprises administering a therapeutically effective amount of a compound or formula (I) or a pharmaceutically acceptable salt thereof to an animal in need thereof. The method is intended to include treating diseases of known viral etiology as well as diseases suspected of being of viral origin including diseases in which the viral pathogen or suspected viral pathogen is unknown.

The components of formula (I) are in accordance with the following structure:

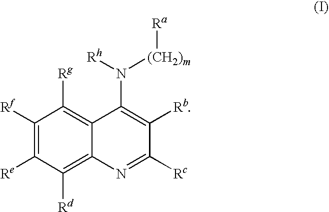

In some embodiments m can be an integer of 0 to 5. In some embodiments, R^{a} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)(CH₂)₀₋₅R^{X}, —C(O)(CH₂)₀₋₅OR^{X}, —OC(O)(CH₂)₀₋₅R^{X}, —C(S)R^{X}, —NHR^{X}, —N═CH—R^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{b}, R^{c}, R^{d}, R^{e}, R^{f}, R^{g} and R^{h}, wherein any member of said strap optionally can be substituted; or R^{e} and R^{b}, R^{c}, R^{d}, R^{e}, R^{f}, R^{g} or R^{h}, together with the atoms to which they are bonded, can form an optionally-substituted aryl, heteroaryl, cyclyl or heterocyclyl.

In some embodiments, R^{b} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{c}, R^{d}, R^{e}, R^{f}, R^{g} and R^{h}, wherein any member of said strap optionally can be substituted; or R^{b} and R^{c}, R^{d}, R^{e}, R^{f}, R^{g} or R^{h}, together with the atoms to which they are bonded, can form a moiety selected from carbocyclyl and heterocyclyl, wherein said moiety can be optionally substituted with one or more R^{X}.

In some embodiments, R^{c} can be selected from the following substituents, or any subset thereof, which include hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{d}, R^{e}, R^{f}, R^{g} and R^{h}, wherein any member of said strap optionally can be substituted; or R^{c} and R^{d}, R^{e}, R^{f}, R^{g} or R^{h}, together with the atoms to which they are bonded, can form a moiety selected from carbocyclyl and heterocyclyl, wherein said moiety can be optionally substituted with one or more R^{X}.

In some embodiments, R^{d} can be selected from the following substituents, or any subset thereof, which include hydrogen, halogen, alkyl, hydroxyalkyl, hydroxyalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl optionally substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{e}, R^{f}, R^{g} and R^{h}, wherein any member of said strap optionally is substituted; or R^{d} and R^{e}, R^{f}, R^{g} or R^{h}, together with the atoms to which they are bonded, can form a moiety selected from carbocyclyl and heterocyclyl, wherein said moiety can be optionally substituted with one or more R^{X}.

In some embodiments, R^{e} can be selected from the following substituents, or any subset thereof, which include hydrogen, halogen, alkyl, hydroxyalkyl, haloalkyl, alkenyl, alkynyl, cycloalkyl, heterocycidalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{f}, R^{g} and R^{h}, wherein any member of said strap optionally is substituted; or R^{e} and R^{f}, R^{g} or R^{h}, together with the atoms to which they are bonded, can form a moiety selected from carbocyclyl and heterocyclyl, wherein said moiety can be optionally substituted with one or more R^{X}.

In some embodiments, R^{f} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{g} and R^{h}, wherein any member of said strap optionally is substituted; or R^{f} and R^{g} or R^{h}, together with the atoms to which they are bonded, can form a moiety selected from carbocyclyl and heterocyclyl, wherein said moiety can be optionally substituted with one or more R^{X}.

In some embodiments, R^{g} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{c}, R^{f} and R^{h}, wherein any member of said strap optionally can be substituted; or R^{g} and R^{h}, together with the atoms to which they are bonded, can form a moiety selected from carbocyclyl and heterocyclyl, wherein said moiety can be optionally substituted with one or more R^{X}.

In some embodiments, R^{h} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}; R^{b}, R^{c}, R^{d}, R^{e}, R^{f} and R^{g}, wherein any member of said strap optionally is substituted.

In various embodiments, each R^{X} can be independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, halogenated aryl, aralkyl, aralkenyl, C(O)O-aryl, O—CH₂-aryl, S(O)(O)—NH-heteroaryl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyl, and alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl; and each pair of R^{Y} and R^{Z}, together with the atoms to which they are bonded, can form an optionally-substituted moiety selected from carbocyclyl and heterocyclyl, wherein said moiety is optionally substituted with one or more R^{X}.

In some embodiments, R^{b} and R^{c}, together with the atoms to which they are bonded, can form a single ring carbocyclyl, wherein said single ring carbocyclyl is optionally substituted with one or more R^{X}. The single ring carbocyclyl can be selected from cycloalkenyl and aryl. In some embodiments, R^{d} and R^{e}, together with the atoms to which they are bonded, can form a single ring carbocyclyl, wherein said single ring carbocyclyl is optionally substituted with one or more R^{X}. The single ring carbocyclyl can be selected from cycloalkenyl and aryl. According to another aspect of the invention, m can be equal to 0, and R^{a} can be a moiety selected from straight or branched hydroxyalkyl containing 1 to 6 carbon atoms. According to yet another aspect of the invention, R^{b}, R^{c}, R^{d}, R^{e} and R^{f} can be independently selected from halogen, —OR^{X}, —C(O)OR^{X} and alkyl. In yet another aspect, m can be equal to 0, and R^{a} can be a moiety selected from aryl optionally substituted with one or more R^{X} and aryl optionally substituted with R^{Y} and R^{Z}. In yet another aspect, m can be equal to 1, and R^{a} can be a moiety selected from furyl optionally substituted with one or more R^{X} and furyl optionally substituted with R^{Y} and R^{Z}, and R^{b} can be —OC(O)R^{X}.

According to yet another aspect of the invention, compounds useful in this invention include those having the formulae of Compounds 1 through 79 listed in Table 2 below. In various embodiments of the invention, particular compounds useful in the invention are represented by the following formulae:

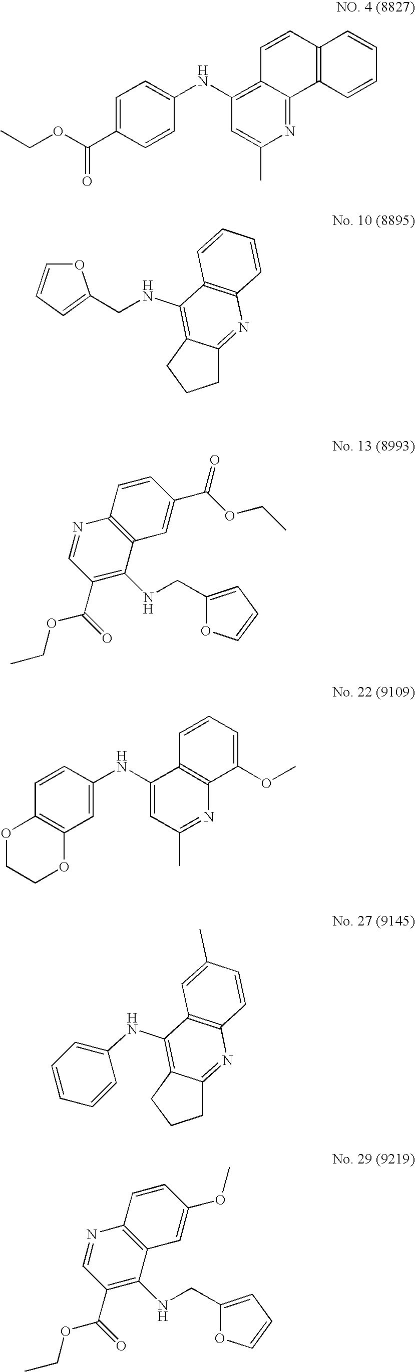

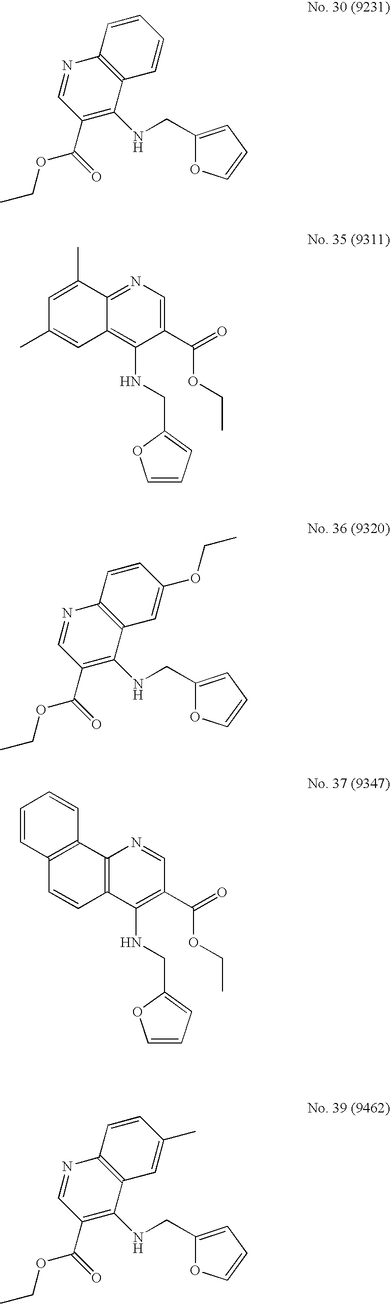

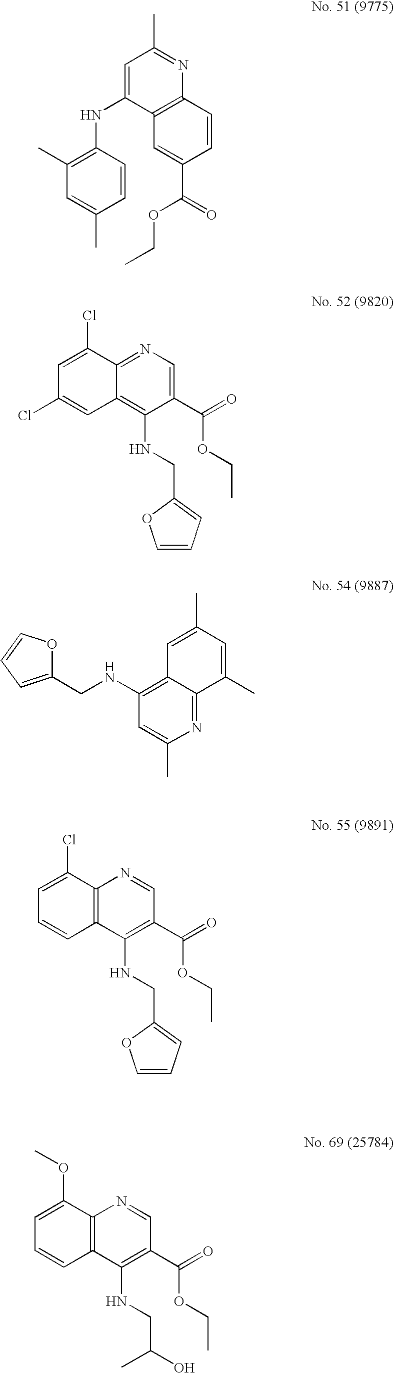

Any combination of the above compounds can be useful in the invention.

In accordance with a further aspect of the invention, the viral infection is caused by an RNA virus. In various embodiments, the viral infection is caused by a positive-strand RNA virus. The virus can be from a virus family selected from the group consisting of Picornaviridae, Caliciviridae, Astroviridae, Coronaviridae, Togaviridae, and Flaviviridae. In particular, the virus can be selected from the group consisting of Sindbis virus (SINV), rubella virus, hepatitis C virus (HCV), West Nile virus (WNV), yellow fever virus (YFV), tick-borne encephalitis virus, Japanese encephalitis virus, coxsackievirus, enterovirus, hepatitis A virus, severe acute respiratory syndrome virus, astrovirus virus, Dengue fever virus (DNG), poliovirus virus, Venezuela encephalitis virus, Western equine encephalomyelitis virus, Eastern equine encephalomyelitis, O'nyong nyong virus, Ross River virus, Chikungunya virus, Rhinovirus, feline calicivirus, murine calicivirus, Norwalk virus, bovine viral diarrhea virus, human coronavirus, Semliki Forest virus, Kunjin virus, Omsk hemorrhagic fever virus, Murray Valley enciphalitis virus, Kyasanur Forest disease virus, Rocio virus, and Astrovirus. In some embodiments, the virus is hepatitis C virus. In some embodiments, the virus is West Nile virus. In some embodiments, the virus is yellow fever virus.

In various embodiments, the infection can be caused by a negative-strand RNA virus. The virus can be from a virus family selected from the group consisting of Paramyxoviridae, Rhabdoviridae, Filoviridae, Orthomyxoviridae, Bunyaviridae, Bomaviridae, and Arenaviridae. In particular, the virus can be selected from the group consisting of respiratory syncytial virus (RSV), Ebola virus (EBOV), rabies virus, Lassa fever virus, La Crosse virus, Rift Valley fever virus, Hantaan virus, California encephalitis virus, influenza virus A, influenza virus B, measles virus, mumps virus, Marburg virus, Bolivian hemorrhagic fever virus, human parainfluenza virus, human metapneumovirus, Nipah virus, Hendra virus, vesicular stomatitis virus, lymphocytic choriomeningitis virus, Junin virus, Bunyamwera virus, Uukuniemi virus, and Crimean-Congo hemorrhagic fever virus. In some embodiments, the virus can be respiratory syncytial virus.

In various embodiments, the viral infection can be caused by a double-strand RNA virus. The virus can be from the Reoviridae virus family. In particular, the virus can be Colorado tick fever.

In accordance with yet another aspect of the invention, the viral infection can be caused by a DNA virus. In various embodiments, the viral infection can be caused by a partial-complex DNA virus. The virus can be from the Hepadanviridae virus family. In particular, the virus can be Hepatitis B virus.

In various embodiments, the viral infection can be caused by a single-strand DNA virus. The virus can be from the Paravavoviridae virus family. In particular, the virus can be human parvovirus.

In various embodiments, the viral infection can be caused by a double-strand DNA virus. The virus can be from a virus family selected from the group consisting of Papillomaviridae, Polyomaviridae, and Herpesviridae. In particular, the virus can be selected from the group consisting of human papillomavirus, JC virus, BK virus, herpes simplex virus 1, herpes simplex virus 2, herpes simplex virus 6, herpes simplex virus 7, herpes simplex virus 8, Eptstein Barr virus, and human cytomegalovirus.

In various embodiments, the virus can be a respiratory virus. In particular, the virus can be selected from the group consisting of parainfluenza virus, human metapneumovirus, rhinovirus, and hantavirus. In some embodiments, the virus can be an enteric virus. In some embodiments, the virus can be selected from the group consisting of enterovirus, rotavirus, and calicivirus.

In various embodiments, the virus can be an encephalitis-ausing virus. In particular, the virus can be selected from the group consisting of West Nile virus and tick-borne encephalitis virus.

In various embodiments, the virus can be a hemorrhagic fever virus. In particular, the virus can be selected from the group consisting of Ebola virus, Marburg virus, and Lassa fever virus.

In various embodiments, the method above further comprises administering a second antiviral agent to the animal. In particular, the second antiviral agent can be selected from the group consisting of interferon and ribavirin. In some embodiments, the animal of the method above can be other than human.

In accordance with another aspect of the invention, a pharmaceutical compound is provided for treating a viral infection in an animal, wherein the compound can comprise a therapeutically effective amount of an aminoquinoline compound or a pharmaceutically acceptable salt thereof; and the aminoquinoline compound corresponds in structure to formula (I):

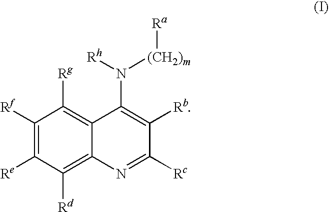

In some embodiments m can be an integer of 0 to 5. In some embodiments, R^{a} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)(CH₂)₀₋₅R^{X}, —C(O)(CH₂)₀₋₅OR^{X}, —OC(O)(CH₂)₀₋₅R^{X}, —C(S)R^{X}, —NHR^{X}, —N═CH—R^{X}, —OR^{X}, —SR^{X}, —O(PO₂)OR^{X}, —N(R^{X})₂, —(CH₂)₀₋₅(N)(R^{Y})(R^{Z}), —OC(R^{W})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{b}, R^{c}, R^{d}, R^{e}, R^{f}, R^{g} and R^{h}, wherein any member of said strap optionally is substituted; or R″ and R^{b}, R^{c}, R^{d}, R^{e}, R^{f}, R^{g} or R^{h}, together with the atoms to which they are bonded, can form an optionally-substituted aryl, heteroaryl, cyclyl or heterocyclyl.

In some embodiments, R^{b} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{c}, R^{d}, R^{e}, R^{f}, R^{g} and R^{h}, wherein any member of said strap optionally is substituted; or R^{b} and R^{c}, R^{d}, R^{e}, R^{f}, R^{g} or R^{h}, together with the atoms to which they are bonded, can form a moiety selected from carbocyclyl and heterocyclyl, wherein said moiety is optionally substituted with one or more R^{X}.

In some embodiments, R^{c} can be selected from the following substituents, or any subset thereof, which include hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{d}, R^{e}, R^{f}, R^{g} and R^{h}, wherein any member of said strap optionally is substituted; or R^{c} and R^{d}, R^{e}, R^{f}, R^{g} or R^{h}, together with the atoms to which they are bonded, can form a moiety selected from carbocyclyl and heterocyclyl, wherein said moiety is optionally substituted with one or more R^{X}.

In some embodiments, R^{d} can be selected from the following substituents, or any subset thereof, which include hydrogen, halogen, alkyl, hydroxyalkyl, hydroxyalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl optionally substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{e}, R^{f}, R^{g} and R^{h}, wherein any member of said strap optionally is substituted; or R^{d} and R^{e}, R^{f}, R^{g} or R^{h}, together with the atoms to which they are bonded, can form a moiety selected ^{from} carbocyclyl and heterocyclyl, wherein said moiety is optionally substituted with one or more R^{X}.

In some embodiments, R^{e} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{e}, R^{d}, R^{f}, R^{g} and R^{h}, wherein any member of said strap optionally is substituted; or R^{e} and R^{f}, R^{g} or R^{h}, together with the atoms to which they are bonded, can form a moiety selected from carbocyclyl and heterocyclyl, wherein said moiety is optionally substituted with one or more R^{X}.

In some embodiments, R^{f} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{g} and R^{h}, wherein any member of said strap optionally is substituted; or R^{f} and R^{g} or R^{h}, together with the atoms to which they are bonded, can form a moiety selected from carbocyclyl and heterocyclyl, wherein said moiety is optionally substituted with one or more R^{X}.

In some embodiments, R^{g} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R²), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{h}, wherein any member of said strap optionally is substituted; or R^{g} and R^{h}, together with the atoms to which they are bonded, can form a moiety selected from carbocyclyl and heterocyclyl, wherein said moiety is optionally substituted with one or more R^{X}.

In some embodiments, R^{h} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{g}, wherein any member of said strap optionally is substituted.

In various embodiments, each R^{X} can be independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, halogenated aryl, aralkyl, aralkenyl, C(O)O-aryl, O—CH₂-aryl, S(O)(O)—NH-heteroaryl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyl, and alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl; and each pair of R^{Y} and R^{Z}, together with the atoms to which they are bonded, can form an optionally-substituted moiety selected from carbocyclyl and heterocyclyl, wherein said moiety is optionally substituted with one or more R^{X}.

In various embodiments, the compound can be effective for treating a viral infection in an animal.

In various embodiments, a kit is provided comprising a pharmaceutical composition described above and, optionally, appropriate diluents, administration devices, and instructions therefor package in a container.

These and other features, aspects and advantages of the present invention will become better understood with reference to the following description, examples and appended claims.

### DETAILED DESCRIPTION

### Abbreviations and Definitions

To facilitate understanding of the invention, a number of terms and abbreviations as used herein are defined below as follows.

2-fused-ring: As used herein, the term “2-fused-ring” heterocyclyl (alone or in combination with another term(s)) refers to a saturated, non-aromatic partially-saturated, or heteroaryl containing two fused rings. Such heterocyclyls include, for example, benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, imidazolopyridazyl, quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, pyrindinyl, isoindolyl, indoleninyl, pyrazolopyrimidinyl, pyrazolopyrazinyl, pyrazolopyridazyl, benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, 4H-quinolizinyl, benzoxazinyl, and benzoisoxazinyl. In some embodiments, the 2-fused-ring heterocyclyls include benzofuranyl, isobenzofuranyl, benzoxazolyl, benzoisoxazolyl, anthranilyl, benzothienyl, isobenzothienyl, benzothiazolyl, benzothiadiazolyl, indolizinyl, pyranopyrrolyl, benzoxadiazolyl, indolyl, isoindazolyl, benzoimidazolyl, benzotriazolyl, purinyl, quinolinyl, isoquinolinyl, pyridopyridinyl, phthalazinyl, quinoxalinyl, benzodiazinyl, pteridinyl, pyrindinyl, isoindolyl, indoleninyl, benzodioxolyl, benzodioxanyl, tetrahydroisoquinolinyl, 4H-quinolizinyl, benzoxazinyl, and benzoisoxazinyl.

Alkyl: As used herein, the term “alkyl” (alone or in combination with another term(s)) refers to a straight- or branched-chain saturated hydrocarbyl substituent typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, octyl, and the like.

Alkenyl: As used herein, the term “alkenyl” (alone or in combination with another term(s)) refers to a straight- or branched-chain hydrocarbyl substituent containing one or more double bonds and typically from 1 to about 20 carbon atoms, more typically from about 2 to about 20 carbon atoms, even more typically from about 2 to about 8 carbon atoms, and still even more typicallyfrom about 2 to about 6 carbon atoms. Examples of such substituents include ═CH₂, ethenyl(vinyl): 2-propenyl; 3-propenyl; 1,4-pentadienyl; 1,4-butadienyl; 1-butenyl; 2-butenyl; 3-butenyl; decenyl; and the like.

Alkoxy: As used herein, the term “alkoxy” (alone or in combination with another term(s)) refers to an alkylether substituent, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

Alkoxycarbonyl: As used herein, the term “alkoxycarbonyl” (alone or in combination with another term(s)) refers to —C(O)—O-alkyl. For example, “ethoxycarbonyl” may be depicted as:

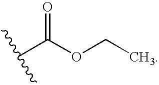

Alkylcarbonyl: As used herein, the term “alkylcarbonyl” (alone or in combination with another term(s)) refers to —C(O)-alkyl. For example, “ethylcarbonyl” may be depicted as:

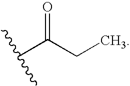

Alkynyl: As used herein, the term “alkynyl” (alone or in combination with another term(s)) refers to a straight- or branched-chain hydrocarbyl substituent containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, 3-butynyl, and the like.

Amino: As used herein, the term “amino” (alone or in combination with another term(s)) refers to —NH₂. The term “monosubstituted amino” (alone or in combination with another term(s)) means an amino substituent wherein a non-hydrogen substituent is in the place of one of the hydrogens. The term “disubstituted amino” (alone or in combination with another term(s)) means an amino substituent wherein non-hydrogen substituents (which may be identical or different) are in the place of both of the hydrogens.

Aminoalkylcarbonyl: As used herein, the term “aminoalkylcarbonyl” (alone or in combination with another term(s)) refers to —C(O)-alkyl-NH₂. For example, “aminomethylcarbonyl” may be depicted as:

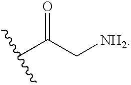

Aminocarbonyl: As used herein, the term “aminocarbonyl” (alone or in combination with another term(s)) refers to —C(O)—NH₂, which also may be depicted as:

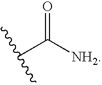

Aminosulfonyl: As used herein, the term “aminosulfonyl” (alone or in combination with another term(s)) refers to —S(O)₂—NH₂, which also may be depicted as:

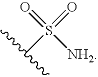

Aryl: As used herein, the term “aryl” (alone or in combination with another term(s)) refers to an aromatic carbocyclyl typically containing from 6 to 14 carbon ring atoms. Examples of aryls include phenyl, naphthalenyl, and indenyl.

Carbocyclyl: As used herein, the term “carbocyclyl” (alone or in combination with another term(s)) refers to a saturated cyclic (i.e., “cycloalkyl”), partially saturated cyclic (i.e., “cycloalkenyl”), or completely unsaturated (i.e., “aryl”) hydrocarbyl substituent typically containing from 3 to 14 carbon ring atoms (“ring atoms” are the atoms bound together to form the ring or rings of a cyclic substituent). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be multiple (typically 2 or 3) rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

Carbonyl: As used herein, the term “carbonyl” (alone or in combination with another term(s)) refers to —C(O)—, which also may be depicted as:

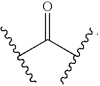

This term also is intended to encompass a hydrated carbonyl substituent, i.e., —C(OH)₂—.

CC₅₀: As used herein, the term “CC₅₀” refers to a standard of measure indicating the concentration of a compound that causes 50 percent of maximum cytotoxicity.

Compound: As used herein, the term “compound” refers to one or more molecules of a substance, wherein a molecule is the smallest particle of a substance that retains the chemical and physical properties of the substance and is composed of two or more atoms.

Comprise, Comprises or Comprising: With reference to the use of the words “comprise” or “comprises” or “comprising” (including the claims), unless the context requires otherwise, those words are used on the basis and clear understanding that they are to be interpreted inclusively, rather than exclusively.

Cyano: As used herein, the term 4“cyano” (alone or in combination with another term(s)) refers to —CN, which also may be depicted:

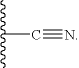

Cycloalkyl: As used herein, the term “cycloalkyl” (alone or in combination with another term(s)) refers to a saturated cyclic hydrocarbyl substituent typically containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentyl”), and cyclohexyl (or “cyclohexyl”). A cycloalkyl alternatively may be multiple (typically 2 or 3) carbon rings fused together, such as, decalinyl or norpinanyl.

Defective genome: As used herein, the term “Defective genome” refers to a DNA or RNA molecule that contains all the genetic elements (e.g., cis-acting elements) required for viral genomic replication and transcription, but lacks one or more of the genetic elements that encode the borrowed factors or enzymes (e.g., trans-acting factors) required for replication. Defective genomes require the addition of a missing factor in order to replicate.

EC₅₀: As used herein, the term “EC₅₀” refers to a standard measure of effective concentration (EC) which is the concentration of a compound required to achieve a 50 percent inhibition of replication of the virus, e.g., a reduction of 50 percent of the replication achieved in the absence of the compound. Sometimes used interchangeably with IC₅₀.

Formula of a Compound: As used herein, the term “Formula of a compound” refers to the chemical formula of the one or more molecules of the substance of the compound;

Halo: As used herein, the prefix “halo” indicates that the substituent to which the prefix is attached is substituted with one or more independently selected halogens. For example, haloalkyl means an alkyl substituent having a halogen in the place of a hydrogen, or multiple halogens in the place of the same number of hydrogens. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein a halogen is in the place of a hydrogen, or multiple halogens are in the place of the same number of hydrogens. Examples of haloalkoxy substituents include chloromethoxy, 1-bromoethoxy, fluoromethoxy, difluoromethoxy, trifluoromethoxy (also known as “perfluoromethyloxy”), 1,1,1-trifluoroethoxy, and the like. It should be recognized that if a substituent is substituted by more than one halogen, those halogens may be identical or different (unless otherwise stated).

Halogen: As used herein, the term “halogen” (alone or in combination with another term(s)) refers to a fluorine substituent (“fluoro,” which may be depicted as —F), chlorine substituent (“chloro,” which may be depicted as —Cl), bromine substituent (“bromo,” which may be depicted as —Br), or iodine substituent (“iodo,” which may be depicted as —I). In various embodiments, fluoro or chloro is provided, particularly fluoro.

Heteroaryl: As used herein, the term “heteroaryl” (alone or in combination with another term(s)) refers to an aromatic heterocyclyl typically containing from 5 to 14 ring atoms. A heteroaryl may be a single ring or multiple (typically 2 or 3) fused rings. Such moieties include, for example, 5-membered rings such as furanyl, thienyl, oxazolyl, isoxazolyl, thiazolyl, isothiazolyl, thiodiazolyl, oxadiazolyl, pyrrolyl, pyrazolyl, imidazolyl, triazolyl, tetrazolyl, oxathiazolyl, and oxatriazolyl; 6-membered rings such as pyridinyl, pyrazinyl, pyrimidinyl, pyridazinyl, triazinyl, and oxathiazinyl; 7-membered rings such as oxepinyl and thiepinyl; 6/5-membered fused-ring systems such as benzofuranyl, isobenzofuranyl, benzoxazolyl, benzoisoxazolyl, anthranilyl, benzothienyl, isobenzothienyl, benzothiazolyl, benzoisothiazolyl, benzothiadiazolyl, indolizinyl, pyranopyrrolyl, benzoxadiazolyl, indolyl, isoindazolyl, benzoimidazolyl, benzotriazolyl, purinyl, imidazopyrazinyl, and imidazolopyridazyl; and 6/6-membered fused-ring systems such as quinolinyl, isoquinolinyl, pyridopyridinyl, phthalazinyl, quinoxalinyl, benzodiazinyl, pteridinyl, pyridazinotetrazinyl, pyrazinotetrazinyl, pyrimidinotetrazinyl, benzoimidazothiazolyl, carbazolyl, and acridinyl. In some embodiments, the 5-membered rings include furanyl, thienyl, oxazolyl, isoxazolyl, thiazolyl, isothiazolyl, oxadiazolyl, pyrazolyl, and imidazolyl; the 6-membered rings include pyridinyl, pyrazinyl, pyrimidinyl, pyridazinyl, and triazinyl; the 6/5-membered fused-ring systems include benzoxazolyl, benzoisoxazolyl, anthranilyl, benzothienyl, isobenzothienyl, and purinyl; and the 6/6-membered fused-ring systems include quinolinyl, isoquinolinyl, and benzodiazinyl.

Heterocyclyl: As used herein, the term “heterocyclyl” (alone or in combination with another term(s)) refers to a saturated (i.e., “heterocycloalkyl”), non-aromatic partially-saturated (i.e., “heterocycloalkenyl”), or heterocyclic aromatic (i.e., “heteroaryl”) ring structure typically containing a total of from 3 to 20 (more typically from 3 to 14) ring atoms. At least one of the ring atoms is a heteroatom (typically oxygen, nitrogen, or sulfur), with the remaining ring atoms being independently selected from the group typically consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, thienyl (also known as “thiophenyl” and “thiofuranyl”), oxazolyl, isoxazolyl, thiazolyl, isbthiazolyl, thiodiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), pyrrolyl, pyrazolyl, imidazolyl, triazolyl, tetrazolyl, oxathiazolyl, oxatriazolyl (including 1,2,3,4-oxatriazolyl and 1,2,3,5-oxatriazolyl), pyridinyl, diazinyl (including pyridazinyl (also known as “1,2-diazinyl”), pyrimidinyl (also known as “1,3-diazinyl”), and pyrazinyl (also known as “1,4-diazinyl”)), triazinyl (including s-triazinyl (also known as “1,3,5-triazinyl”), as-triazinyl (also known 1,2,4-triazinyl), and v-triazinyl (also known as “1,2,3-triazinyl”)), oxathiazinyl (including 1,2,5-oxathiazinyl and 1,2,6-oxathiazinyl), oxepinyl, thiepinyl, dihydrofuranyl, tetrahydrofuranyl, dihydrothienyl (also known as “dihydrothiophenyl”), tetrahydrothienyl (also known as “tetrahydrothiophenyl”), isopyrrolyl, pyrrolinyl, pyrrolidinyl, isoimidazolyl, imidazolinyl, imidazolidinyl, pyrazolinyl, pyrazolidinyl, dithiolyl, oxathiolyl, oxathiolanyl, oxazolidinyl, isoxazolidinyl, thiazolinyl, isothiazolinyl, thiazolidinyl, isothiazolidinyl, dioxazolyl (including 1,2,3-dioxazolyl, 1,2,4-dioxazolyl, 1,3,2-dioxazolyl, and 1,3,4-dioxazolyl), pyranyl (including 1,2-pyranyl and 1,4-pyranyl), dihydropyranyl, tetrahydropyranyl, piperidinyl, piperazinyl, oxazinyl (including 1,2,3-oxazinyl, 1,3,2-oxazinyl, 1,3,6-oxazinyl (also known as “pentoxazolyi”), 1,2,6-oxazinyl, and 1,4-oxazinyl), isoxazinyl (including o-isoxazinyl and p-isoxazinyl), oxadiazinyl (including 1,4,2-oxadiazinyl and 1,3,5,2-oxadiazinyl), morpholinyl, azepinyl, and diazepinyl.

A heterocyclyl alternatively may be from 2 to 5 (more typically from 2 or 3) rings fused together, such as, for example, indolizinyl, pyranopyrrolyl, purinyl, imidazopyrazinyl, imidazolopyridazyl, pyridopyridinyl (including pyrido[3,4-b]-pyridinyl, pyrido[3,2-b]-pyridinyl, pyrido[4,3-b]-pyridinyl, and naphthyridinyl), pteridinyl, pyridazinotetrazinyl, pyrazinotetrazinyl, pyrimidinotetrazinyl, pyrindinyl, pyrazolopyrimidinyl, pyrazolopyrazinyl, pyrazolopyridazyl, or 4H-quinolizinyl. In some embodiments, the multi-ring heterocyclyls are indolizinyl, pyranopyrrolyl, purinyl, pyridopyridinyl, pyrindinyl, and 4H-quinolizinyl.

Other examples of fused-ring heterocyclyls include benzo-fused heterocyclyls, such as, for example, benzofuranyl (also known as “coumaronyl”), isobenzofuranyl, benzoxazolyl, benzoisoxazolyl (also known as “indoxazinyl”), anthranilyl, benzothienyl (also known as “benzothiophenyl,” “thionaphthenyl,” and “benzothiofuranyl”), isobenzothienyl (also known as “isobenzothiophenyl,” “isothionaphthenyl,” and “isobenzothiofuranyl”), benzothiazolyl, benzoisothiazolyl, benzothiadiazolyl, benzoxadiazolyl, indolyl, isoindazolyl (also known as “benzpyrazolyl”), benzoimidazolyl, benzotriazolyl, benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzoimidazothiazolyl, carbazolyl, acridinyl, isoindolyl, indoleninyl (also known as “pseudoindolyl”), benzodioxolyl, chromanyl, isochromanyl, thiochromanyl, isothiochromanyl, chromenyl, isochromenyl, thiochromenyl, isothiochromenyl, benzodioxanyl, tetrahydroisoquinolinyl, benzoxazinyl (including 1,3,2-benzoxazinyl, 1,4,2-benzoxazinyl, 2,3,1-benzoxazinyl, and 3,1,4-benzoxazinyl), benzoisoxazinyl (including 1,2-benzisoxazinyl and 1,4-benzisoxazinyl), benzoxadiazinyl, and xanthenyl. In some embodiments, the benzo-fused heterocyclyls are benzofuranyl, isobenzofuranyl, benzoxazolyl, benzoisoxazolyl, anthranilyl, benzothienyl, isobenzothienyl, benzothiazolyl, benzothiadiazolyl, benzoxadiazolyl, indolyl, isoindazolyl, benzoimidazolyl, benzotriazolyl, benzazinyl, phthalazinyl, quinoxalinyl, benzodiazinyl, carbazolyl, acridinyl, isoindolyl, indoleninyl, benzodioxolyl, chromanyl, isochromanyl, thiochromanyl, benzodioxanyl, tetrahydroisoquinolinyl, benzoxazinyl, benzoisoxazinyl, and xanthenyl.

Hydrogen: As used herein, the term “hydrogen”, (alone or in combination with another term(s)) refers to a hydrogen substituent (or “hydrido”), and may be depicted as —H.

Hydroxy: As used herein, the term “hydroxy” (alone or in combination with another term(s)) refers to —OH.

IC₅₀: As used herein, the term “IC₅₀” refers to a standard of measure of inhibitory concentration (IC) which is the concentration of a compound required to achieve a 50 percent inhibition of viral replication. IC₅₀ is often used as interchangeable with EC50.

Keto or Oxo: As used herein, the terms “keto” or “oxo” (alone or in combination with another term(s)) refer to an oxo substituent, and may be depicted as ═O or as —C(O)—.

Nitro: As used herein, the term “nitro” (alone or in combination with another term(s)) refers to —NO₂.

Oxy: As used herein, the term “oxy” (alone or in combination with another term(s)) refers to an ether substituent, and may be depicted as —O—.

Perhalo: As used herein, the prefix “perhalo” indicates that a halogen is in the place of each hydrogen on the substituent to which the prefix is attached. If all the halogens are identical, the prefix typically will identify the halogen. Thus, for example, the term “perfluoro” means that a fluoro is in the place of each hydrogen on the substituent to which the prefix is attached. To illustrate, the term “perfluoroalkyl” means an alkyl substituent wherein a fluoro is in the place of each hydrogen. Examples of perfluoroalkyl substituents include trifluoromethyl (—CF₃), perfluorobutyl, perfluoroisopropyl, perfluorododecyl, perfluorodecyl, and the like. To illustrate further, the term “perfluoroalkoxy” means an alkoxy substituent wherein a fluoro is in the place of each hydrogen. Examples of perfluoroalkoxy substituents include trifluoromethoxy (—O—CF₃), perfluorobutoxy, perfluoroisopropoxy, perfluorododecoxy, perfluorodecoxy, and the like.

Pharmaceutically Acceptable: As used herein, the term “pharmaceutically acceptable” is used adjectivally to mean that the modified, noun is appropriate for use as a pharmaceutical product or as a part of a pharmaceutical product. Generally, something is pharmaceutically acceptable if it does not cause an unacceptable adverse or allergic reaction when administered to the subject.

Substitutions: As used herein, the term “substitutable” refers to a compound having a substituent comprising at least one carbon, nitrogen, oxygen, or sulfur atom that is bonded to one or more hydrogen atoms. Thus, for example, hydrogen, halogen, and cyano do not fall within this definition. If a substituent is described as being “substituted,” a non-hydrogen substituent is in the place of a hydrogen on a carbon, nitrogen, oxygen, or sulfur of the substituent. Thus, for example, a substituted alkyl substituent is an alkyl substituent wherein at least one non-hydrogen substituent is in the place of a hydrogen on the alkyl substituent. To illustrate, monofluoroalkyl is alkyl substituted with a fluoro, and difluoroalkyl is alkyl substituted with two fluoros. It should be recognized that if there are more than one substitutions on a substituent, each non-hydrogen substituent may be identical or different (unless otherwise stated).

If a substituent is described as being “optionally substituted,” the substituent is either (1) substituted, or (2) not substituted. When the members of a group of substituents are described generally as being optionally substituted, any atom capable of substitution in each member of such group may be (1) substituted, or (2) not substituted. Such a characterization contemplates that some members of the group are not substitutable. Atoms capable of substitution include, for example, carbon bonded to at least one hydrogen, oxygen bonded to at least one hydrogen, sulfur bonded to at least one hydrogen, or nitrogen bonded to at least one hydrogen. On the other hand, hydrogen alone, halogen, oxo, and cyano do not fall within the definition of being capable of substitution.

Sulfonyl: As used herein, the term “sulfonyl” (alone or in combination with another term(s)) refers to —S(O)₂—, which also may be depicted as:

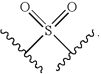

Thus, for example, “alkyl-sulfonyl-alkyl” means alkyl-S(O)₂-alkyl.

Sulfoxido: As used herein, the term “sulfoxido” (alone or in combination with another term(s)) refers to —S(O)—, which also may be depicted as:

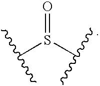

Thus, for example, “alkyl-sulfoxido-alkyl” means alkyl-S(O)-alkyl.

Therapeutic Index (TI): As used herein, the term “therapeutic index” refers to a ratio of cell cytotoxic concentration (CC) over the effective concentration (EC). For example, TI=CC₅₀/EC₅₀.

Thio or Thia: As used herein, the terms “thio” or “thia” (alone or in combination with another term(s)) refer to a thiaether substituent, i.e., an ether substituent wherein a divalent sulfur atom is in the place of the ether oxygen atom. Such a substituent may be depicted as —S—. This, for example, “alkyl-thio-alkyl” means alkyl-5-alkyl.

Thiocarbonyl: The term “thiocarbonyl” (alone or in combination with another term(s)) refers to a carbonyl wherein a sulfur is in the place of the oxygen. Such a substituent may be depicted as —C(S)—, and also may be depicted as:

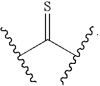

Thiol or Mercapto: As used herein, the terms “thiol” or “mercapto” (alone or in combination with another term(s)) refer to a sulfhydryl substituent, and may be depicted as —SH.

A carbocyclyl or heterocyclyl can optionally be substituted with, for example, one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, —OH, —C(O)—OH, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with, for example, one or more halogen. The aryl and cycloalkyl portions of such optional substituents are typically single-rings containing from 3 to 6 ring atoms, and more typically from 5 to 6 ring atoms.

An aryl or heteroaryl can optionally be substituted with, for example, one or more substituents independently selected from the group consisting of halogen, —OH, —CN, —NO₂, —SH, —C(O)—OH, amino, aminoalkyl, alkyl, alkylthio, carboxyalkylthio, alkylcarbonyloxy, alkoxy, alkoxyalkyl, alkoxycarbonylalkoxy, alkoxyalkylthio, alkoxycarbonylalkylthio, carboxyalkoxy, alkoxycarbonylalkoxy, carbocyclyl, carbocyclylalkyl, carbocyclyloxy, carbocyclylthio, carbocyclylalkylthio, carbocyclylamino, carbocyclylalkylamino, carbocyclylcarbonylamino, carbocyclylalkyl, carbocyclylcarbonyloxy, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyloxy, heterocyclyloxyalkoxyheterocyclyl, heterocyclylthioalkylthioheterocyclyl, heterocyclylthioalkoxyheterocyclyl, and heterocyclyloxyalkylthioheterocyclyl. More typically, an aryl or heteroaryl may, for example, optionally be substituted with one or more substituents independently selected from the group consisting of halogen, —OH, —CN, —NO₂, —SH, —C(O)—OH, amino, amino-C₁-C₆-alkyl, C₁-C₆-alkyl, C₁-C₆alkylthio, carboxy-C₁-C₆-alkylthio, C₁-C₆-alkylcarbonyloxy, C₁-C₆alkoxy, C₁-C₆-alkoxy-C₁-C₆-alkyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, aryl-C₁-C₆-alkyl, aryloxy, arylthio, aryl-C₁-C₆-alkylthio, arylamino, aryl-C₁-C₆-alkylamino, arylcarbonylamino, arylcarbonyloxy, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyloxy, heteroaryl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, and heteroarylcarbonyloxy. Here, one or more hydrogens bound to a carbon in any such substituent may, for example, optionally be replaced with halogen. In addition, any cycloalkyl, aryl, and heteroaryl portions of such optional substituents are typically single-rings containing 3 to 6 ring atoms, and more typically 5 or 6 ring atoms.

A prefix attached to a multi-component substituent only applies to the first component. To illustrate, the term ‘alkylcycloalkyl’ contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkoxyalkyl indicates that only the alkoxy component of the alkoxyalkyl substituent is substituted with one or more halogens. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other selected substituent(s).

When words are used to describe a substituent, the rightmost-described component of the substituent is the component that has the free valence. To illustrate, benzene substituted with methoxyethyl has the following structure:

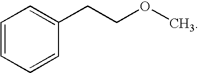

As can be seen, the ethyl is bound to the benzene, and the methoxy is the component of the substituent that is the component furthest from the benzene. As further illustration, benzene substituted with cyclohexylthiobutoxy has the following structure:

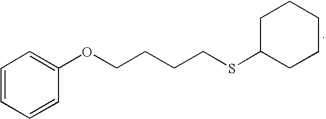

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexylethyl, then the chemical would be X-ethyl-cyclohexyl-methyl-Y.

When a chemical formula is used to describe a mono-valent substituent, the dash on the left side of the formula indicates the portion of the substituent that has the free valence. To illustrate, benzene substituted with —C(O)—OH has the following structure:

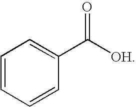

When a chemical formula is used to describe a di-valent (or “linking”) element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

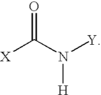

In some instances, the number of carbon atoms in a hydrocarbyl substituent (e.g., alkyl, alkenyl, alkynyl, or cycloalkyl) is indicated by the prefix “Cₓ—C_{y}—”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

### Aminoquinoline Compounds

In accordance with this invention, Applicants have discovered that aminoquinoline compounds tend to be effective for inhibiting viral activity (particularly, RNA virus activity, such as HCV, RSV, WNV, YFV, DNG, EBOV and SINV activity).

As noted above, the compounds used in the present invention can generally correspond in structure to Formula I:

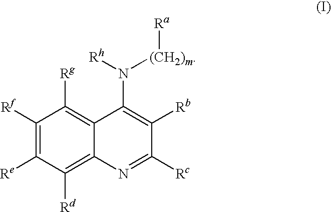

In this formula, m can be an integer from 0 to 5, and R^{a}, R^{b}, R^{c}, R^{e}, R^{f}, R^{g} and R^{h} are defined as follows:

Each of R^{a}, R^{b}, R^{c}, R^{e}, R^{f}, R^{g} and R^{h} can be either an independent substituent or forms part of a ring structure. More specifically, the substituent compounds of Formula I can be described as follows.

In some embodiments, R^{a} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)(CH₂)₀₋₅R^{X}, —C(O)(CH₂)₀₋₅OR^{X}, —OC(O)(CH₂)₀₋₅R^{X}, —C(S)R^{X}, —NHR^{X}, —N═CH—R^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{b}, R^{c}, R^{d}, R^{e}, R^{f}, R^{g} and R^{h}, wherein any member of said strap optionally is substituted. In various embodiments, R^{a} and R^{b}, R^{c}, R^{d}, R^{e}, R^{f}, R^{g} or R^{h}, together with the atoms to which they are bonded, can form an optionally-substituted aryl, heteroaryl, cyclyl or heterocyclyl.

In various embodiments, each R^{X} can be independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, halogenated aryl, aralkyl, Aralkenyl, C(O)O-aryl, O—CH₂-aryl, S(O)(O)—NH-heteroaryl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyl, and alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl.

In various embodiments, each pair of R^{Y} and R^{Z}, together with the atoms to which they are bonded, can form an optionally-substituted moiety selected from carbocyclyl and heterocyclyl, wherein said moiety is optionally substituted with one or more R^{X}.

In some embodiments, R^{b} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{c}, R^{d}, R^{e}, R^{f}, R^{g} and R^{h}, wherein any member of said strap optionally is substituted. In various embodiments, R^{b} and R^{c}, R^{d}, R^{e}, R^{f}, R^{g} or R^{h}, together with the atoms to which they are bonded, can form a moiety selected from carbocyclyl and heterocyclyl, wherein said moiety is optionally substituted with one or more R^{X}. R^{X}, R^{Y} and R^{Z} can be as defined above.

In some embodiments, R^{c} can be selected from the following substituents, or any subset thereof, which include hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{d}, R^{e}, R^{f}, R^{g} and R^{h}, wherein any member of said strap optionally is substituted. In various embodiments, R^{c} and R^{d}, R^{e}, R^{f}, R^{g} or R^{h}, together with the atoms to which they are bonded, can form a moiety selected from carbocyclyl and heterocyclyl, wherein said moiety is optionally substituted with one or more R^{X}. R^{X}, R^{Y} and R^{Z} are as defined above.

In some embodiments, R^{d} can be selected from the following substituents, or any subset thereof, which include hydrogen, halogen, alkyl, hydroxyalkyl, hydroxyalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl optionally substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{e}, R^{f}, R^{g} and R^{h}, wherein any member of said strap optionally is substituted. In various embodiments, R^{d} and R^{e}, R^{f}, R^{g} or R^{h}, together with the atoms to which they are bonded, can form a moiety selected from carbocyclyl and heterocyclyl, wherein said moiety is optionally substituted with one or more R^{X}. R^{X}, R^{Y} and R^{Z} can be as defined above.

In some embodiments, R^{e} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}; aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{f}, R^{g} and R^{h}, wherein any member of said strap optionally is substituted. In various embodiments, R^{e} and R^{f}, R^{g} or R^{h}, together with the atoms to which they are bonded, can form a moiety selected from carbocyclyl and heterocyclyl, wherein said moiety is optionally substituted with one or more R^{X}. R^{X}, R^{Y} and R^{Z} can be as defined above;

In some embodiments, R^{f} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{g} and R^{h}, wherein any member of said strap optionally is substituted. In various embodiments, R^{f} and R^{g} or R^{h}, together with the atoms to which they are bonded, can form a moiety selected from carbocyclyl and heterocyclyl, wherein said moiety is optionally substituted with one or more R^{X}. R^{X}, R^{Y} and R^{Z} can be as defined above.

In some embodiments, R^{g} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{h}, wherein any member of said strap optionally is substituted. In various embodiments, R^{g} and R^{h}, together with the atoms to which they are bonded, can form a moiety selected from carbocyclyl and heterocyclyl, wherein said moiety is optionally substituted with one or more R^{X}. R^{X}, R^{Y} and R^{Z} can be as defined above.

In some embodiments, R^{h} can be selected from the following substituents, or any subset thereof, which include hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{g}, wherein any member of said strap optionally is substituted. R^{X}, R^{Y} and R^{Z} can be as defined above.

In various embodiments, each R^{X} can be independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, halogenated aryl, aralkyl, aralkenyl, C(O)O-aryl, O—CH₂-aryl, S(O)(O)—NH-heteroaryl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyl, and alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl; and each pair of R^{Y} and R^{Z}, together with the atoms to which they are bonded, can form an optionally-substituted moiety selected from carbocyclyl and heterocyclyl, wherein said moiety is optionally substituted with one or more R^{X}.

In various embodiments, the compounds useful in the invention can correspond to the structural formulae of Table 2 below:

TABLE 2
Compounds of the Invention

| No. | I.D. | Formula | M.W. | Structure |
|---|---|---|---|---|
| 1. | 8749 | C₂₃H₁₆N₂ | 320.387 | 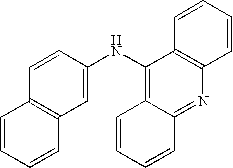 |
| 2. | 8770 | C₂₁H₁₈N₂ | 298.381 | 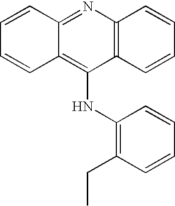 |
| 3. | 8804 | C₁₇H₁₆N₂ | 248.322 | 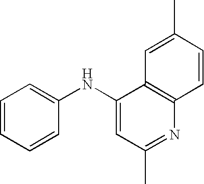 |
| 4. | 8827 | C₂₃H₂₀N₂O₂ | 356.417 | 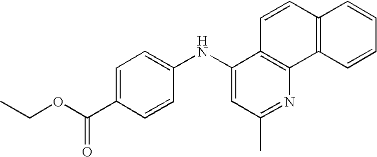 |
| 5. | 8835 | C₁₉H₁₅N₃ | 285.343 | 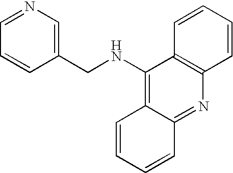 |
| 6. | 8856 | C₁₈H₁₈N₂ | 262.349 | 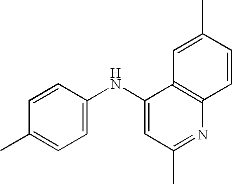 |
| 7. | 8864 | C₂₁H₁₈N₂O | 314.38 | 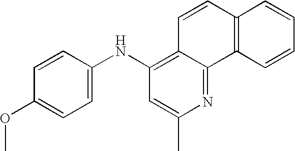 |
| 8. | 8868 | C₂₀H₁₈N₂O₂ | 318.369 | 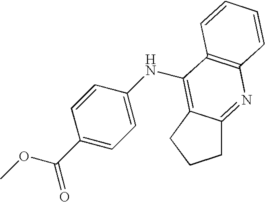 |
| 9. | 8874 | C₂₁H₁₈N₂ | 298.381 | 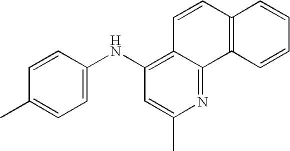 |
| 10. | 8895 | C₁₇H₁₆N₂O | 264.322 | 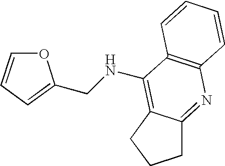 |
| 11. | 8902 | C₁₉H₁₈N₂ | 274.36 | 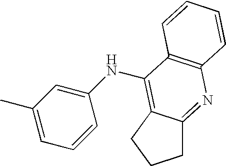 |
| 12. | 8961 | C₁₅H₁₆N₂ | 224.301 | 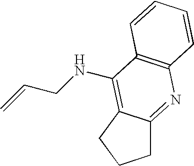 |
| 13. | 8993 | C₂₀H₂₀N₂O₅ | 368.383 | 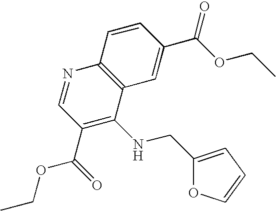 |
| 14. | 8998 | C₁₈H₁₅ClN₂O₂ | 326.777 | 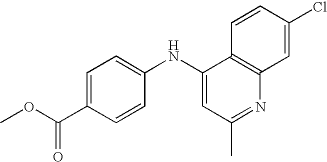 |
| 15. | 9047 | C₁₇H₂₂N₂ | 254.37 | 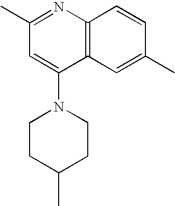 |
| 16. | 9088 | C₁₉H₁₈N₂O | 290.359 | 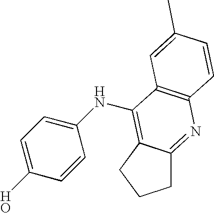 |
| 17. | 9095 | C₁₈H₁₆N₂O | 276.332 | 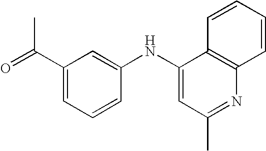 |
| 18. | 9096 | C₁₈H₁₈N₂ | 262.349 | 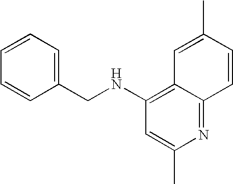 |
| 19. | 9104 | C₁₇H₁₄Cl₂N₂O₃ | 365.211 | 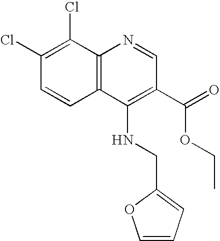 |
| 20. | 9105 | C₁₇H₁₆N₂O | 264.322 | 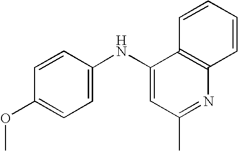 |
| 21. | 9108 | C₂₃H₂₅N₃O | 359.464 | 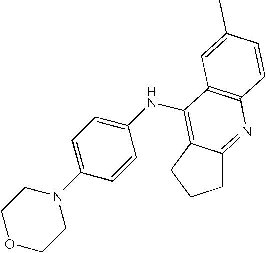 |
| 22. | 9109 | C₁₉H₁₈N₂O₃ | 322.358 | 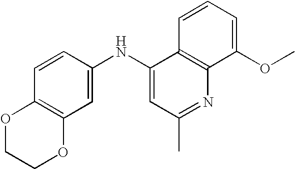 |
| 23. | 9116 | C₁₆H₁₆N₂O | 252.311 | 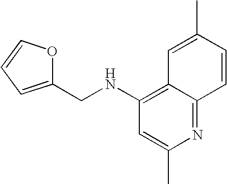 |
| 24. | 9121 | C₁₉H₂₀N₂O | 292.375 | 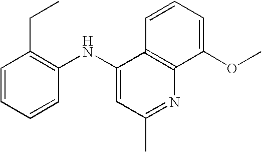 |
| 25. | 9141 | C₁₇H₁₅N₃O₃ | 309.319 | 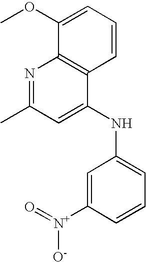 |
| 26. | 9144 | C₂₀H₁₇F₃N₂ | 342.358 | 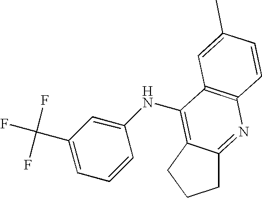 |
| 27. | 9145 | C₁₉H₁₈N₂ | 274.36 | 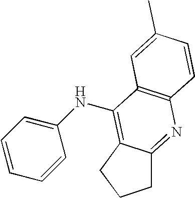 |
| 28. | 9155 | C₂₀H₂₀N₂ | 288.386 | 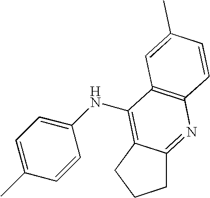 |
| 29. | 9219 | C₁₈H₁₈N₂O₄ | 326.347 | 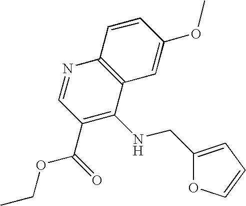 |
| 30. | 9231 | C₁₇H₁₆N₂O₃ | 296.321 | 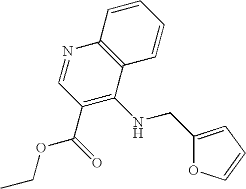 |
| 31. | 9278 | C₂₃H₂₅N₃O₄ | 407.462 | 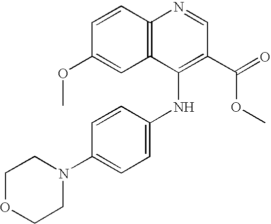 |
| 32. | 9279 | C₂₀H₁₉lN₂O₃ | 462.281 | 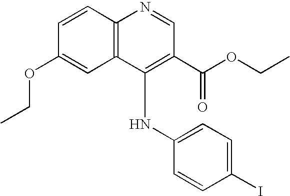 |
| 33. | 9291 | C₂₀H₁₉N₃O₄ | 365.383 | 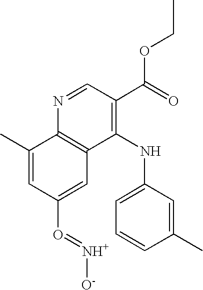 |
| 34. | 9304 | C₂₂H₂₄N₂O₃ | 364.438 | 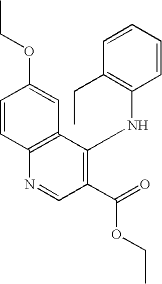 |
| 35. | 9311 | C₁₉H₂₀N₂O₃ | 324.374 | 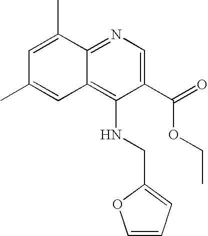 |
| 36. | 9320 | C₁₉H₂₀N₂O₄ | 340.373 | 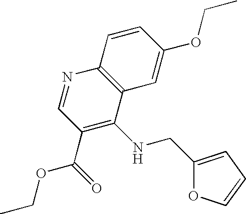 |
| 37. | 9347 | C₂₁H₁₈N₂O₃ | 346.379 | 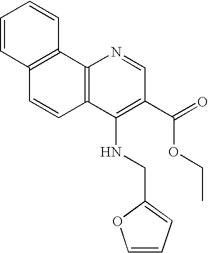 |
| 38. | 9457 | C₂₃H₂₅N₃O₃ | 391.463 | 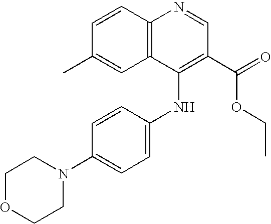 |
| 39. | 9462 | C₁₈H₁₈N₂O₃ | 310.347 | 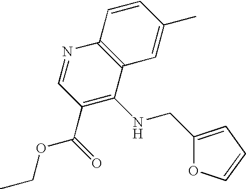 |
| 40. | 9604 | C₁₇H₁₂ClF₃N₂ | 336.739 | 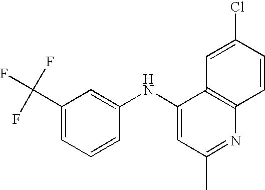 |
| 41. | 9605 | C₁₈H₁₇BrN₂O | 357.244 | 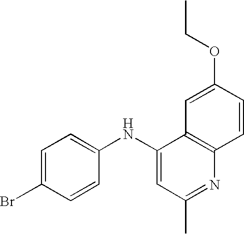 |
| 42. | 9615 | C₂₅H₂₃ClN₂O₂ | 418.915 | 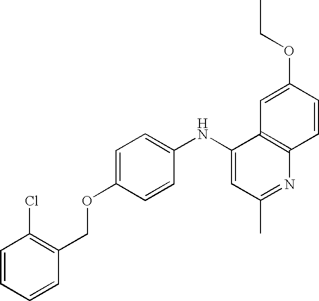 |
| 43. | 9634 | C₂₄H₂₁ClN₂O₂ | 404.889 | 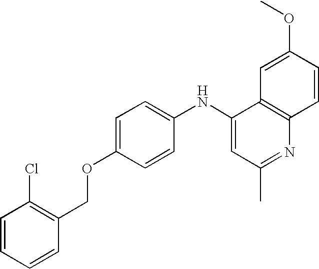 |
| 44. | 9645 | C₂₅H₂₄N₂O | 368.471 | 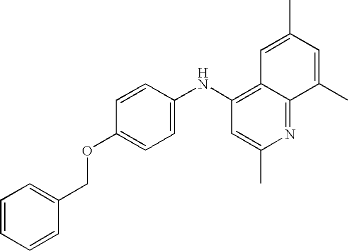 |
| 45. | 9664 | C₁₉H₁₉ClN₂O | 326.82 | 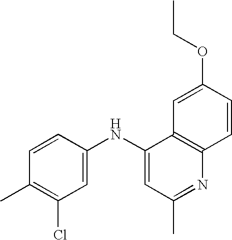 |
| 46. | 9668 | C₁₈H₁₈N₂O₃ | 310.347 | 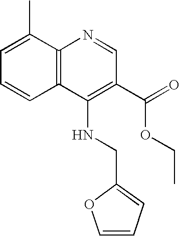 |
| 47. | 9686 | C₁₈H₁₇ClN₂ | 296.794 | 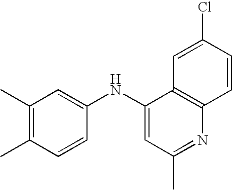 |
| 48. | 9716 | C₂₀H₂₀N₂O | 304.386 | 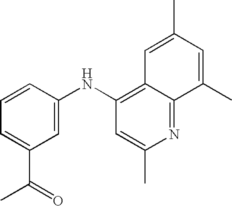 |
| 49. | 9723 | C₁₈H₁₈N₂O | 278.348 | 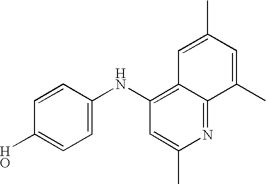 |
| 50. | 9725 | C₁₈H₁₇ClN₂ | 296.794 | 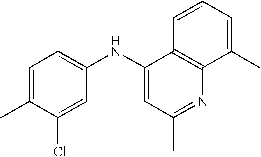 |
| 51. | 9775 | C₂₁H₂₂N₂O₂ | 334.412 | 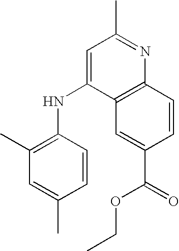 |
| 52. | 9820 | C₁₇H₁₄Cl₂N₂O₃ | 365.211 | 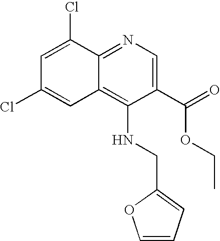 |
| 53. | 9880 | C₁₈H₁₆Cl₂N₂ | 331.239 | 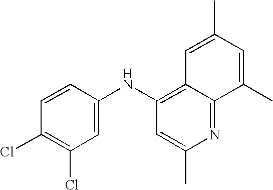 |
| 54. | 9887 | C₁₇H₁₈N₂O | 266.338 | 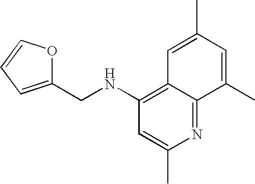 |
| 55. | 9891 | C₁₇H₁₅ClN₂O₃ | 330.766 | 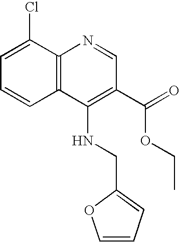 |
| 56. | 9923 | C₂₀H₂₂N₂O₃ | 338.4 | 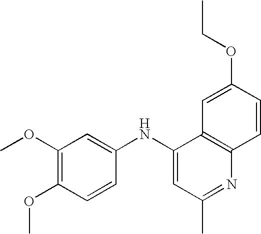 |
| 57. | 9943 | C₂₀H₂₃N₃O | 321.416 | 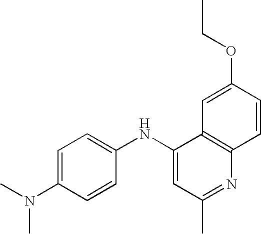 |
| 58. | 9958 | C₁₉H₁₉ClN₂O₂ | 342.819 | 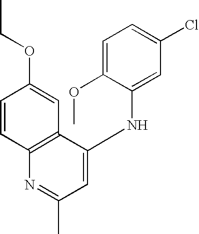 |
| 59. | 10006 | C₁₈H₁₇ClN₂O | 312.793 | 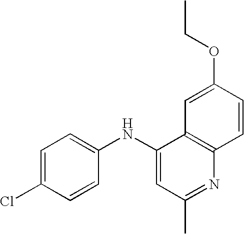 |
| 60. | 10643 | C₁₈H₁₅ClN₂O₂ | 326.777 | 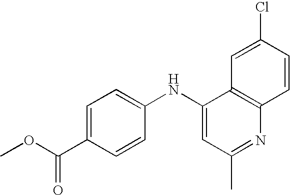 |
| 61. | 10651 | C₁₉H₁₈N₂O₂ | 306.358 | 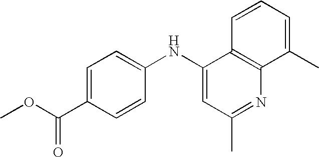 |
| 62. | 25608 | C₂₀H₂₀N₂O₃ | 336.384 | 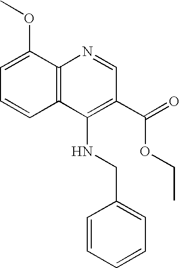 |
| 63. | 25612 | C₂₃H₂₄N₂O₄ | 392.448 | 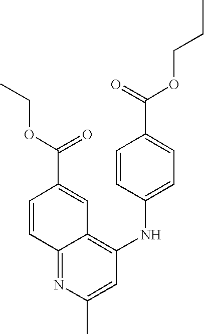 |
| 64. | 25650 | C₂₀H₂₁Cl₃N₂O₆ | 491.75 | 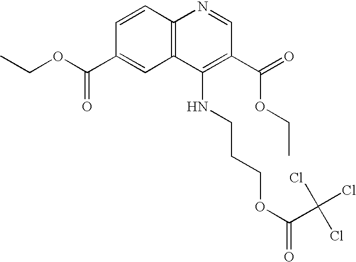 |
| 65. | 25653 | C₁₈H₁₇ClN₂ | 296.794 | 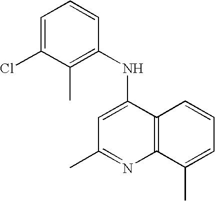 |
| 66. | 25755 | C₁₇H₂₂FN₃O₂ | 319.374 | 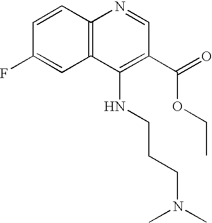 |
| 67. | 25765 | C₁₈H₁₈N₂O₄ | 326.347 | 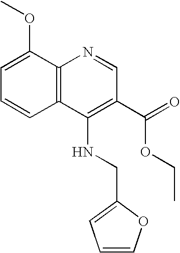 |
| 68. | 25775 | C₁₉H₁₉N₃O₃ | 337.372 | 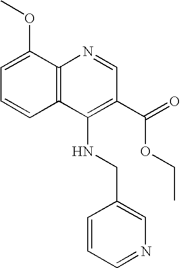 |
| 69. | 25784 | C₁₆H₂₀N₂O₄ | 304.341 | 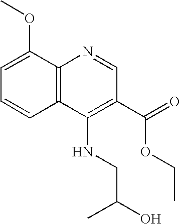 |
| 70. | 25793 | C₁₆H₂₀N₂O₄ | 304.341 | 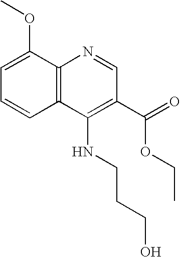 |
| 71. | 25796 | C₂₁H₂₂N₂O₃ | 350.411 | 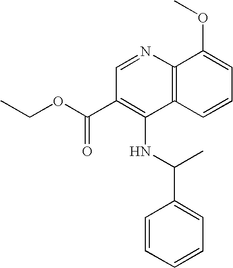 |
| 72. | 25839 | C₂₅H₂₂F₃N₅O₄S | 545.533 | 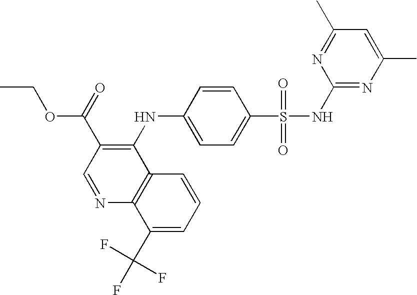 |
| 73. | 25846 | C₁₈H₁₆N₂O₃ | 308.331 | 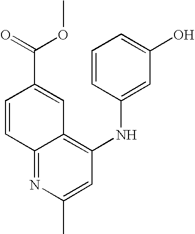 |
| 74. | 25863 | C₂₄H₂₂FN₅O₄S | 495.526 | 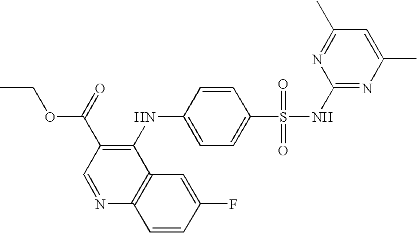 |
| 75. | 25888 | C₁₉H₂₇N₃O₂ | 329.437 | 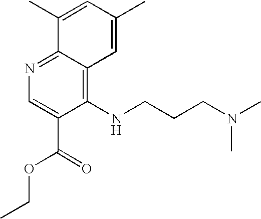 |
| 76. | 25975 | C₁₇H₁₅IN₂O | 390.218 | 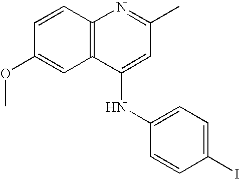 |
| 77. | 80058 | C₁₉H₂₁N₃O | 307.39 | 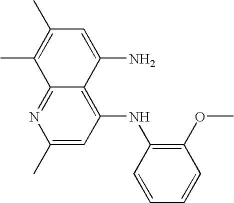 |
| 78. | 80086 | C₁₈H₁₇N₃O₂ | 307.346 | 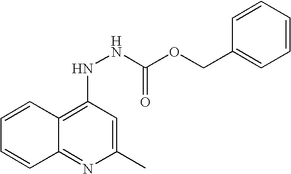 |
| 79. | 80087 | C₁₉H₂₁N₃O | 255.143 | 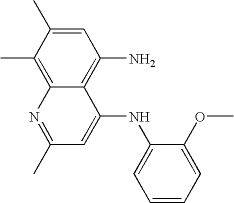 |

More particularly, compounds useful in the invention correspond to the following structural formulae (including the compound number and a unique identifier in parentheses):

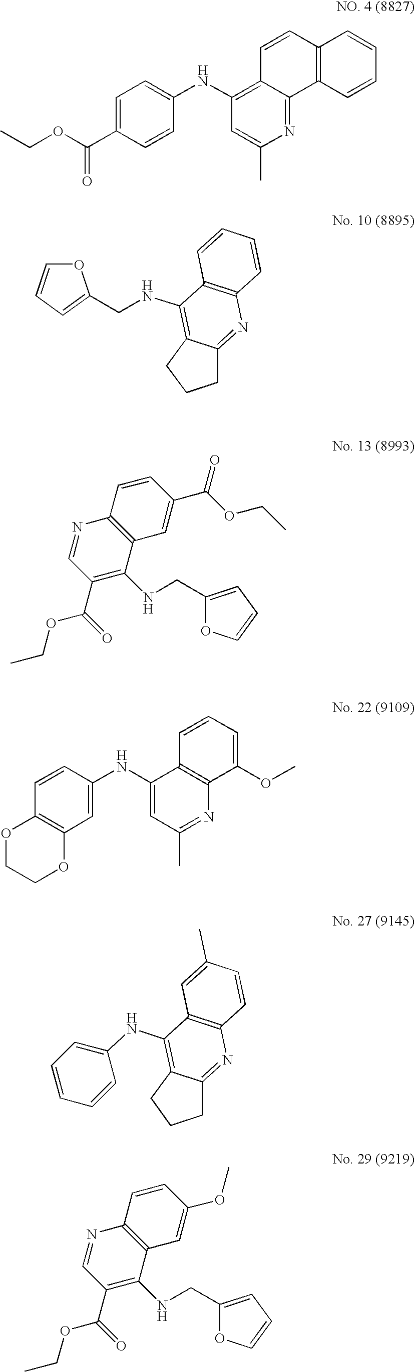

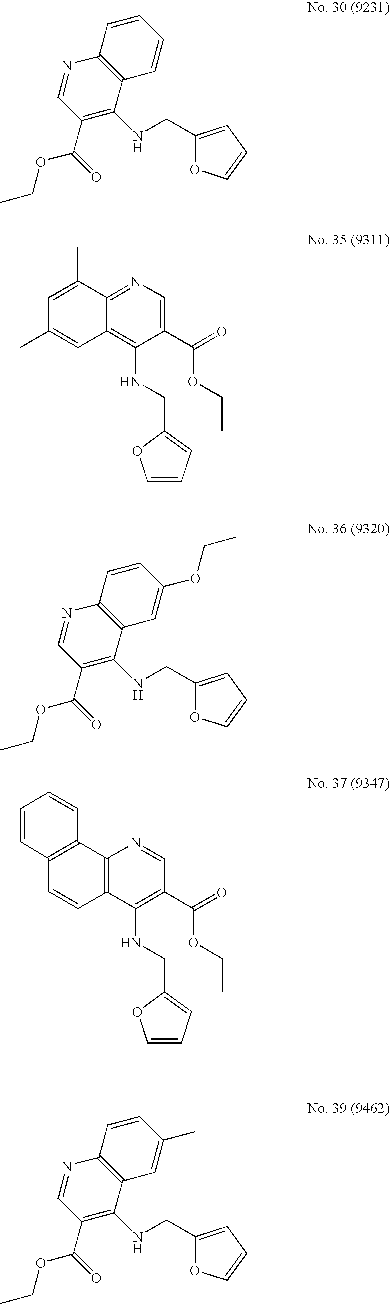

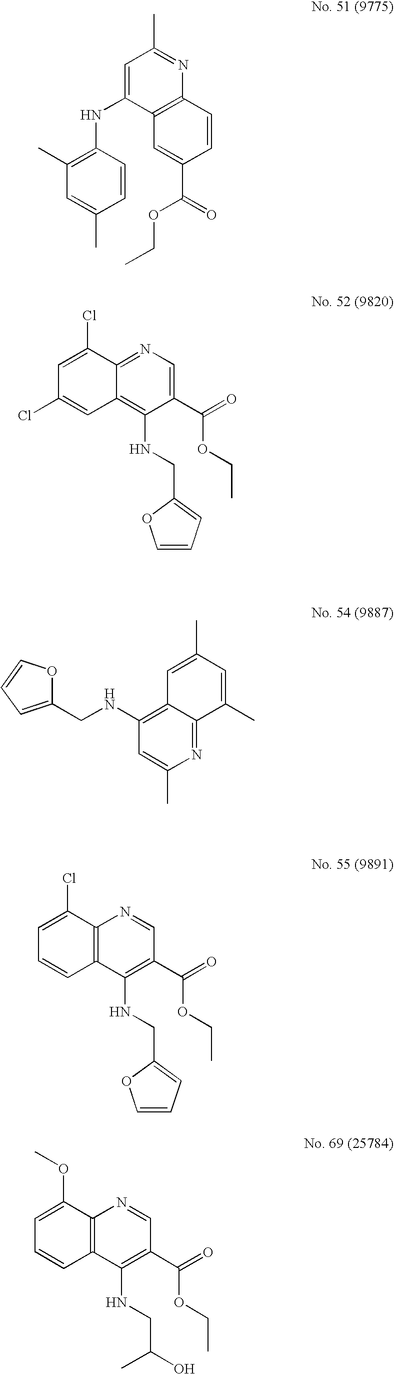

Those of skill in the art will recognize that combinations of the above compounds are useful in the invention.

Compounds useful with this invention generally may be prepared by methods, known in the art. The compounds of this invention can be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

When a salt is intended to be administered to a subject (as opposed to, for example, being used in an in vitro context), the salt may be pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically acceptable acid addition salts of the compounds of this invention may be prepared from an inorganic or organic acid. Examples of suitable inorganic acids include hydrochloric, hydrobromic, hydroiodic, nitric, carbonic, sulfuric, and phosphoric. Suitable organic acids generally include, for example, aliphatic, cycloaliphatic, aromatic, araliphatic, heterocyclic, carboxylic, and sulfonic classes of organic acids. Specific examples of suitable organic acids include acetate, trifluoroacetate, formate, propionate, succinate, glycolate, gluconate, digluconate, lactate, malate, tartaric, citrate, ascorbate, glucuronate, maleate, fumarate, pyruvate, aspartate, glutamate, benzoate, anthranilic, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic, β-hydroxybutyric, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically acceptable base addition salts of the compounds of this invention include, for example, metallic salts and organic salts. Various metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Various organic salts can be made from amines, such as tromethamine, diethylamine, N,N′-dibenzylethylenediamine, chloroprocaine, diethanolamine, ethylenediamine, meglumine (N-methylglucamine), and procaine. Basic nitrogen-containing groups can be quaternized with materials such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), aralkyl halides (e.g., benzyl and phenethyl bromides), and others. In particular embodiments, the salt comprises a hydrochloric acid (HCl) salt.

### Treatment of Virus-Related Conditions

This invention is directed, in part, to a method fortreating a pathological condition caused (directly or indirectly) by viral activity. Animals benefiting from such a method generally include, for example, humans. The method, however, may be used in veterinary contexts as well to treat other mammals, such as other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.). It is contemplated that the method may further be used in other veterinary contexts to treat, for example, birds, reptiles, fish, and amphibians.

In some embodiments, the condition is associated with an RNA virus.

In some embodiments, the condition is associated with a positive-strand RNA virus. Such viruses include, for example, viruses falling within a viral family selected from the group consisting of Picornaviridae, Caliciviridae, Astroviridae, Coronaviridae, Togaviridae, and Flaviviridae. Specific examples of positive-strand RNA viruses include Sindbis virus, rubella virus, hepatitis C virus (HCV), West Nile virus (WNV), yellow fever virus (YFV), tick-bome encephalitis (TBE) virus, Japanese encephalitis virus, coxsackievirus, enterovirus, hepatitis A virus, Severe acute respiratory syndrome (SARS) virus, astrovirus, Dengue fever virus (DV), poliovirus, Venezuela encephalitis virus (VEE), Western equine encephalomyelitis (WEE) virus, Eastern equine encephalomyelitis (EEE) virus, O'nyong nyong virus, Ross River virus, Chikungunya virus, Rhinovirus, feline calicivirus, murine calicivirus, Norwalk virus, bovine viral diarrhea virus (BVDV), human coronavirus, Semliki Forest virus, Kunjin virus, Omsk hrmorrhagic fever (Omsk HF) virus, Murray Valley enciphalitis virus, Kyasanur Forest disease virus, Rocio virus, and Astrovirus.

In some embodiments, the condition is associated with hepatitis C virus.

In some embodiments, the condition is associated with West Nile virus.

In some embodiments, the condition is associated with yellow fever virus.

In some embodiments, the condition is associated with a negative-strand RNA virus. Such viruses include, for example, viruses falling within a viral family selected from the group consisting of Paramyxoviridae, Rhabdoviridae, Filoviridae, Orthomyxoviridae, Bunyaviridae, Bornaviridae, and Arenaviridae. Specific examples of negative-strand RNA viruses include respiratory syncytial virus (RSV), Ebola virus, rabies virus, Lassa fever virus, La Crosse virus, Rift Valley fever virus, Hantaan virus, California encephalitis virus, influenza virus A, influenza virus B, measles virus, mumps virus, Marburg virus, Bolivian hemorrhagic fever (Bolivian HF) virus, human parainfluenza virus (HPIV), human metapneumovirus (hMPV), Nipah virus, Hendra virus (equine morbillivirus), vesicular stomatitis virus (VSV), lymphocytic choriomeningitis (LCM) virus, Junin virus (Argentine hemorrhagic fever virus or Argentine HF virus), Bunyamwera virus, Uukuniemi virus, and Crimean-Congo hemorrhagic fever (CCHF) virus.

In some embodiments, the condition is associated with respiratory syncytial virus.

In some embodiments, the condition is associated with double-strand RNA virus. In some such embodiments, the virus is from the Reoviridae virus family. Examples of double-strand RNA viruses include Colorado tick fever.

In some emboments, the condition is associated with a DNA virus.

In some embodiments, the condition is associated with a partial-complex DNA virus. In some such embodiments, the virus is from the Hepadanviridae virus family. Examples of partial-complex DNA viruses include Hepatitis B virus.

In some embodiments, the condition is associated with a single-strand DNA virus. In some such embodiments, the virus is from the Paravavoviridae virus family. Examples of single-strand DNA viruses include human parvovirus.

In some embodiments, the condition is associated with a double-strand DNA virus. Such viruses include, for example, viruses falling within a viral family selected from the group consisting of Papillomaviridae, Polyomaviridae, and Herpesviridae. Specific examples of double-strand DNA viruses include human papillomavirus, JC virus, BK virus, herpes simplex virus 1, herpes simplex virus 2, herpes simplex virus 6, herpes simplex virus 7, herpes simplex virus 8, Eptstein Barr virus, and human cytomegalovirus.

In some embodiments, the condition is associated with a respiratory virus. Such viruses include, for example, parainfluenza viruses, human metapneumovirus, rhinoviruses, and hantaviruses.

In some embodiments, the condition is associated with an enteric virus. Such viruses include, for example, enteroviruses, rotavirus, and caliciviruses.

In some embodiments, the condition is associated with an encephalitis-causing virus. Such viruses include, for example, West Nile virus and tick-bome encephalitis virus.

In some embodiments, the condition is associated with a hemorrhagic fever virus. Such viruses include, for example, Ebola virus, Marburg virus, and Lassa fever virus.

Typically, a compound (or pharmaceutically acceptable salt thereof) described in this patent is administered in a therapeutically-effective amount to a subject suffering from (or prediposed to) a viral infection. Generally, the term “therapeutically-effective amount” means an amount that is effective to inhibit activity of the target virus(es) or effective to treat the targeted condition in a reasonable amount of time. Here, the term “inhibit” means reducing or eliminating the targeted viral activity. And the term “treat” means ameliorating, suppressing, eradicating, preventing, reducing the risk of, or delaying the onset of the targeted condition.

One skilled in the art generally can determine an appropriate dosage. Factors affecting a particular dosage regimen (including the amount of antiviral compound delivered, frequency of administration, and whether administration is continuous or intermittent) include, for example, the type, age, weight, sex, diet, and condition of the subject; the type of pathological condition and its severity; the nature of the desired effect; whether the purpose of administration is prophylactic or to treat an existing viral infection; pharmacological considerations, such as the activity, efficacy, pharmacokinetic, and toxicology profiles of the particular antiviral compound used; the route of administration and whether a drug delivery system is utilized; and whether the antiviral compound is administered as part of a combination therapy (e.g., whether the agent is administered in combination with one or more active compounds, other agents, radiation, etc.).

Compositions for oral administration may be, for example, prepared in a manner such that a single dose in one or more oral preparations contains at least about 20 mg of the antiviral compound per square meter of subject body surface area, or at least about 50, 100, 150, 200, 300, 400, or 500 mg of the antiviral compound per square meter of subject body surface area (the average body surface area for a human is, for example, 1.8 square meters). In particular, a single dose of a composition for oral administration contains from about 20 to about 600 mg (more preferably from about 20 to about 400 mg, even more preferably from about 20 to about 300 mg, and still even more preferably from about 20 to about 200 mg) of the antiviral compound per square meter of subject body surface area. Compositions for parenteral administration are, for example, prepared in a manner such that a single dose contains at least about 20 mg of the antiviral compound per square meter of subject body surface area, or at least about 40, 50, 100, 150, 200, 300, 400, or 500 mg of the antiviral compound per square meter of subject body surface area. Preferably, a single dose in one or more parenteral preparations contains from about 20 to about 500 mg (more preferably from about 20 to about 400, even more preferably from about 20 to about 400 mg, and still even more preferably from about 20 to about 350 mg) of the antiviral compound per square meter of subject body surface area. It should be recognized that these oral and parenteral dosage ranges simply represent generally preferred dosage ranges, and are not intended to limit the invention. The dosage regimen actually employed can vary widely, and, therefore, can deviate from the normal preferred dosage regimen. It is contemplated that one skilled in the art will tailor these ranges to the individual subject.

As indicated above, it is contemplated that the aminoquinoline compounds and salts of this invention may be used as part of a combination therapy. The term “combination therapy” means the administration of two or more therapeutic treatments directed to the pathological condition. In this specification, the pathological condition generally comprises a condition associated (directly or indirectly) with viral activity. The therapeutic treatments of the combination generally may be co-administered in a substantially simultaneous manner. Two active compounds could be co-administered as, for example: (a) a single formulation (e.g., a single capsule) having a fixed ratio of active ingredients; or (b) multiple, separate formulations (e.g., multiple capsules) for each compound. The therapeutic treatments of the combination may alternatively (or additionally) be administered at different times. In either case, the chosen treatment regimen preferably provides beneficial effects of the drug combination in treating the condition. Such a combination therapy may comprise administering an aminoquinoline compound or salt of this invention with, for example, one or more additional aminoquinoline compounds or salts of this invention, cytokines (including interferon, and particularly interferon alpha), ribavirin, nucleoside/tide reverse transcriptase inhibitors (“NRTIs,” these include abacavir (Ziagen), lamivudine, 3TC (Epivir), tenofovir (Viread), abacavir/lamivudine/zidovudine (Trizivir), lamivudine/zidovudine (Combivir), stavudine, d4T (Zerit), didanosine, ddI (Videx, Videx EC), zalcitabine, ddC(HIVID), and zidovudine, and AZT (Retrovir)), protease inhibitors (“PIs,” these include amprenavir (Agenerase), nelfinavir (Viracept), saquinavir (Fortavase), indinavir (Crixivan), ritonavir (Norvir), saquinavir (Invirase), and lopinavir/ritonavir (Kaletra)), non-nucleoside reverse transcriptase inhibitors (“NnRTIs,” these include delavirdine (Rescriptor), efavirenz (Sustiva), and nevirapine (Viramune)), or a combination thereof.

It is further contemplated that the aminoquinoline compounds and salts of this invention can be used in the form of a kit that is suitable for use in performing the treatment methods described above, packaged in a container. The kit can contain the aminoquinoline compound or compounds and, optionally, appropriate diluents, devices or device components suitable for administration and instructions for use in accordance with the methods of the present invention. The devices can include parenteral injection devices, such as syringes or transdermal patch or the like. Device components can include cartridges for use in injection devices and the like. In one embodiment, the kit comprises a first dosage form comprising a aminoquinoline compound or salt of this invention and a second dosage form comprising another active ingredient in quantities sufficient to carry out the methods of the present invention. Preferably, the first dosage form and the second dosage form together comprise a therapeutically-effective amount of the compounds for treating the targeted condition(s).

This invention also is directed, in part, to pharmaceutical compositions (or medicaments) comprising a therapeutically-effective amount of a compound or salt of this invention, as well as processes for making such compositions. Such compositions generally comprise one or more pharmaceutically-acceptable carriers (e.g., excipients, vehicles, auxiliaries, adjuvants, diluents, etc.) and/or other active ingredients. Formulation of these compositions may be achieved by various methods known in the art. A general discussion of these methods may be found in, for example, Hoover, John E., Remington's Pharmaceutical Sciences (Mack Publishing Co., Easton, Pa.: 1975). See also, Liberman, H. A. See also, Lachman, L., eds., Pharmaceutical Dosage Forms (Marcel Decker, New York, N.Y., 1980).

The preferred composition depends on the route of administration. Any route of administration may be used as long as the target of the compound or salt is available via that route. Often suitable routes of administration include, for example, oral, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

Pharmaceutically acceptable carriers that may be used in conjunction with the compounds of this invention are well known to those of ordinary skill in the art. Carriers are selected based on a number of factors including, for example, the particular antiviral compound(s) or salt(s) used; the compound's concentration, stability, and intended bioavailability; the condition being treated; the subject's age, size, and general condition; the route of administration; etc. A general discussion related to carriers may be found in, for example, J. G. Nairn, Remington's Pharmaceutical Science, pp. 1492-1517 (A. Gennaro, ed., Mack Publishing Co., Easton, Pa. (1985)).

Solid dosage forms for oral administration include, for example, capsules, tablets, gelcaps, pills, dragees, troches, powders, granules, and lozenges. In such solid dosage forms, the compounds or salts are ordinarily combined with one or more adjuvants. If administered per os, the compounds or salts can be mixed with lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can, for example, include a coating (e.g., an enteric coating) to delay disintegration and absorption. The concentration of the antiviral compound in a solid oral dosage form is preferably from about 5 and about 50% (more preferably from about 8 to about 40%, and even-more preferably from about 10 to about 30%) by weight based on the total weight of the composition.

Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents. The concentration of the antiviral compound preferably is from about 0.01 to about 10 mg (more preferably from about 0.01 to about 5 mg, even more preferably from about 0.01 to about 1 mg, and still even more preferably from about 0.01 to about 0.5 mg) per ml of the composition. Relatively low concentrations are generally preferred because, many of the antiviral compounds tend to be most soluble at low concentrations.

Techniques for making oral dosage forms useful in the present invention are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

In some embodiments, for example, tablets or powders for oral administration are prepared by dissolving the antiviral compound in a pharmaceutically acceptable solvent capable of dissolving the compound to form a solution and then evaporating when the solution is dried under vacuum. An additional carrier(s) also may be added to the solution before drying. The resulting solution is dried under vacuum to form a glass. The glass is then mixed with a binder to form a powder. This powder may be mixed with fillers or other conventional tableting agents, and then processed to form a tablet. Alternatively, the powder may be added to a liquid carrier to form a solution, emulsion, suspension, or the like.

In some embodiments, solutions for oral administration are prepared by dissolving the antiviral compound in a pharmaceutically acceptable solvent capable of dissolving the compound to form a solution. An appropriate volume of a carrier is added to the solution while stirring to form a pharmaceutically acceptable solution for oral administration.

“Parenteral administration” includes subcutaneous injections, intravenous injections, intraarterial injections, intraorbital injections, intracapsular injections, intraspinal injections, intraperitoneal injections, intramuscular injections, intrasternal injections, and infusion. Dosage forms suitable for parenteral administration include solutions, suspensions, dispersions, emulsions, and any other dosage form that can be administered parenterally.

Injectable preparations (e.g., sterile injectable aqueous, or oleaginous suspensions) can be formulated according to the known art using suitable dispersing, wetting agents, and/or suspending agents. Acceptable vehicles for parenteral use include both aqueous and nonaqueous pharmaceutically-acceptable solvents

Suitable pharmaceutically-acceptable aqueous solvents include, for example, water, saline solutions, dextrose solutions (e.g., such as DW5), electrolyte solutions, etc.

Suitable pharmaceutically-acceptable nonaqueous solvents include, for example, the following (as well as mixtures thereof): alcohols (these include, for example, α-glycerol formal, 3-glycerol formal, 1,3-butyleneglycol, aliphatic or aromatic alcohols having from 2 to about 30 carbons (e.g., methanol, ethanol, propanol, isopropanol, butanol, t-butanol, hexanol, octanol, amylene hydrate, benzyl alcohol, glycerin (glycerol), glycol, hexylene glycol, tetrahydrofurfuranyl alcohol, lauryl alcohol, cetyl alcohol, and stearyl alcohol), fatty acid esters of fatty alcohols (e.g., polyalkylene glycols, such as polypropylene glycol and polyethylene glycol), sorbitan, sucrose, and cholesterol); amides (these include, for example, dimethylacetamide (DMA), benzyl benzoate DMA, dimethylformamide, N-(β-hydroxyethyl)-lactamide, N,N-dimethylacetamide-amides, 2-pyrrolidinone, 1-methyl-2-pyrrolidinone, and polyvinylpyrrolidone); esters (these include, for example, acetate esters (e.g., monoacetin, diacetin, and triacetin), aliphatic and aromatic esters (e.g., ethyl caprylate or octanoate, alkyl oleate, benzyl benzoate, or benzyl acetate), dimethylsulfoxide (DMSO), esters of glycerin (e.g., mono, di, and tri-glyceryl citrates and tartrates), ethyl benzoate, ethyl acetate, ethyl carbonate, ethyl lactate, ethyl oleate, fatty acid esters of sorbitan, glyceryl monostearate, glyceride esters (e.g., mono, di, or tn-glycerides), fatty acid esters (e.g., isopropyl myristrate), fatty acid derived PEG esters (e.g., PEG-hydroxyoleate and PEG-hydroxystearate), N-methylpyrrolidinone, pluronic 60, polyoxyethylene sorbitol oleic polyesters (e.g., poly(ethoxylated)₃₀₋₆₀ sorbitol poly(oleate)₂4 poly(oxyethylene)₁₅₋₂₀ monooleate, poly(oxyethylene)_{15.20} mono 12-hydroxystearate, and poly(oxyethylene)₁₅₋₂₀ mono ricinoleate), polyoxyethylene sorbitan esters (e.g., polyoxyethylene-sorbitan monooleate, polyoxyethylene-sorbitan monopalmitate, polyoxyethylene-sorbitan monolaurate, polyoxyethylene-sorbitan monostearate, and POLYSORBATE 20, 40, 60, and 80 (from ICI Americas, Wilmington, Del.)), polyvinylpyrrolidone, alkyleneoxy modified fatty acid esters (e.g., polyoxyl 40 hydrogenated castor oil and polyoxyethylated castor oils, such as CREMOPHOR EL solution or CREMOPHOR RH 40 solution), saccharide fatty acid esters (i.e., the condensation product of a monosaccharide (e.g., pentoses, such as, ribose, ribulose, arabinose, xylose, lyxose, and xylulose: hexoses, such as glucose, fructose, galactose, mannose, and sorbose; trioses; tetroses; heptoses; and octoses), disaccharide (e.g., sucrose, maltose, lactose, and trehalose), oligosaccharide, or a mixture thereof with one or more C₄-C₂₂ fatty acids (e.g., saturated fatty acids, such as caprylic acid, capric acid, lauric acid, myristic acid, palmitic acid, and stearic acid; and unsaturated fatty acids, such as palmitoleic acid, oleic acid, elaidic acid, erucic acid, and linoleic acid), and steroidal esters); ethers (these are typically alkyl, aryl, and cyclic ethers having from 2 to about 30 carbons. Examples include diethyl ether, tetrahydrofuran, dimethyl isosorbide, diethylene glycol monoethyl ether), and glycofurol (tetrahydrofurfuranyl alcohol polyethylene glycol ether); ketones (these typically have from about 3 to about 30 carbons. Examples include acetone, methyl ethyl ketone, methyl isobutyl ketone); hydrocarbons (these are typically aliphatic, cycloaliphatic, and aromatic hydrocarbons having from about 4 to about 30 carbons. Examples include benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), and tetramethylenesulfoxide); oils (these include oils of mineral, vegetable, animal, essential, or synthetic origin. These include mineral oils, such as aliphatic and wax-based hydrocarbons, aromatic hydrocarbons, mixed aliphatic and aromatic based hydrocarbons, and refined paraffin oil; vegetable oils, such as linseed, tung, safflower, soybean, castor, cottonseed, groundnut, rapeseed, coconut, palm, olive, corn, corn germ, sesame, persic, and peanut oil; glycerides, such as mono-, di-, and triglycerides; animal oils, such as fish, marine, sperm, cod-liver, haliver, squalene, squalane, and shark liver oil; oleic oils; and polyoxyethylated castor oil); alkyl, alkenyl, or aryl halides (these include alkyl or aryl halides having from 1 to about 30 carbons and one or more halogen substituent. Examples include methylene chloride); monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (SOLUTOL HS-15, from BASF, Ludwigshafen, Germany); polyoxyethylene glycerol; sodium laurate; sodium oleate; and sorbitan monooleate. Other pharmaceutically acceptable solvents for use in the invention are well known to those of ordinary skill in the art. General discussion relating to such solvents may be found in, for example, The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics 3d ed., (G. Banker et al., eds., Marcel Dekker, Inc., New York, N.Y. (1995)), The Pharmacological Basis of Therapeutics, (Goodman & Gilman, McGraw Hill Publishing), Pharmaceutical Dosage Forms, (H. Lieberman et al., eds., Marcel Dekker, Inc., New York, N.Y. (1980)), Remington's Pharmaceutical Sciences, 19th ed., (A. Gennaro, ed., Mack Publishing, Easton, Pa., (1995)), The United States Pharmacopeia 24, The National Formulaty 19, (National Publishing, Philadelphia, Pa. (2000)); Spiegel, A. J., et al., “Use of Nonaqueous Solvents in Parenteral Products,” J. Pharma. Sciences, Vol. 52, No. 10, pp. 917-927 (1963).

Preferred solvents include those known to stabilize the antiviral compound(s) or sait(s) of interest. These typically include, for example, oils rich in triglycerides, such as safflower oil, soybean oil, and mixtures thereof; and alkyleneoxy-modified fatty acid esters, such as polyoxyl 40 hydrogenated castor oil and polyoxyethylated castor oils (e.g., CREMOPHOR EL solution or CREMOPHOR RH 40 solution). Commercially available triglycerides include INTRALIPID emulsified soybean oil (Kabi-Pharmacia Inc., Stockholm, Sweden), NUTRALIPID emulsion (McGaw, Irvine, Calif.), LIPOSYN II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), LIPOSYN III 2% emulsion (a 2% fat emulsion solution containing 100 mg safflower oil, 100 mg soybean oil, 12 mg egg phosphafides, and 25 mg glycerin per ml of solution; Abbott Laboratories, Chicago, Ill.), natural or synthetic glycerol derivatives containing the docosahexaenoyl group at levels of from about 25 to about 100% (by weight based on the total fatty acid content) (DHASCO from Martek Biosciences Corp., Columbia, Md.; DHA MAGURO from Daito Enterprises, Los Angeles, Calif.; SOYACAL; and TRAVEMULSION. Ethanol is an often preferred solvent for dissolving the antiviral compound or salt to form solutions, emulsions, and the like.

Additional components can be included in the compositions of this invention for various purposes generally known in the pharmaceutical industry. These components tend to impart properties that; for example, enhance retention of the antiviral compound or salt at the site of administration, protect the stability of the composition, control the pH, facilitate processing of the antiviral compound or salt into pharmaceutical formulations, and the like. Specific examples of such components include cryoprotective agents; agents for preventing reprecipitation of the antiviral compound or salt surface: active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stearyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide etc.); and antioxidants (e.g., sodium metabisulfite, sodium bisulfite, sodium sulfite, dextrose, phenols, thiophenols, etc.).

Techniques and compositions for making parenteral dosage forms are generally known in the art. Formulations for parenteral administration may, for example, be prepared from one or more sterile powders and/or granules having a compound or salt of this invention and one or more of the carriers or diluents mentioned for use in the formulations for oral administration. The powder or granule typically is added to an appropriate volume of a solvent (typically while agitating (e.g., stirring) the solvent) that is capable of dissolving the powder or granule. Preferred solvents include, for example, water, polyethylene glycol, propylene glycol, ethanol, corn oil, cottonseed oil, peanut oil, sesame oil, benzyl alcohol, sodium chloride, and/or various buffers.

Emulsions for parenteral administration can be prepared by, for example, dissolving a compound or salt of this invention in any pharmaceutically acceptable solvent capable of dissolving the compound to form a solution; and adding an appropriate volume of a carrier, which is an emulsion, to the solution while stirring to form the emulsion. Solutions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the solution.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols

“Topical administration” includes the use of transdermal administration, such as transdermal patches or iontophoresis devices.

If desired, the emulsions or solutions described above for oral or parenteral administration can be packaged in IV bags, vials, or other conventional containers in concentrated form, and then diluted with a pharmaceutically acceptable liquid (e.g., saline) to form an acceptable antiviral concentration before use.

Other adjuvants and modes of administration well-known in the pharmaceutical art may also be used.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Compounds and Antiviral Activity

In the following examples, compounds of the present invention were evaluated for antiviral activity against HCV, RSV, WNV, YFV, DNG, EBOV, and SINV.

In conducting the experiments, stock solutions of the antiviral compounds were prepared at a concentration of 10 mM in DMSO. Dilutions were made to screening concentrations in cellular growth media. Compounds were initially tested at 25 μM final concentration in primary screening tests, and subsequently tested at a range of from 75 μM to 0.75 μM final concentration in secondary screening tests. The compounds were added to cells and incubated for 24 hours at 37° C. and 5% CO₂ (v/v).

The methods for measuring antiviral activity and cell toxicity are described below in Examples 2-9. The results of primary screening tests are shown in Table 3 as percent inhibition of viral replication for each of the compounds tested. The results of secondary screening tests are shown in Tables 4 and 5 as EC50, CC₅₀ and TI values. EC₅₀ represents the concentration estimated to produce 50% inhibition of viral replication in the antiviral assays; CC₅₀ represents the concentration estimated to produce 50% cell death in the cell toxicity assay; and TI represents the therapeutic index calculated as the ratio of the CC₅₀ concentration to the EC₅₀. Compounds that were not tested for antiviral activity are indicated in the tables as “NT”.

### Example 2

### Measurement of HCV Antiviral Activity

HCV antiviral activity was measured in a HCV Neo screening assay by determining HCV replicon reduction effects of compounds through NPTII ELISA quantitation of Neomycin Phosphotransferase levels.

Clone A cells, a clone of a human hepatoma-derived cell line (Huh7) which carries constitutively replicating subgenomic hepatits C virus RNA, were plated in 96 well tissue culture treated microplates and allowed to settle for 4 hours at 37° C. and 5% CO₂ (v/v). Test compounds were added to appropriate final concentration with DMSO concentration being held constant at 1% in all wells. No compound controls consisted of cells with media plus DMSO at 1%. Background control wells were cells treated with 150U Interferon alpha in media plus DMSO at 1%. Cells were incubated in the presence of compound for 24 hours at 37° C. and 5% CO₂ (v/v).

Neomycin Phosphotransferase II protein was quantified using NPTII ELISA assay kit (Agdia, Inc. of Elkhart, Ind., USA). Media and compound were removed from cells. Cell lysates were prepared by addition of 1× extraction buffer and shaking vigorously for 15 min. Lysates were then transferred to NPTII capture plate and shaken vigorously for 2 hours. Capture plates were washed with PBST 8 times. Plates were filled with conjugated antibody and shaken vigorously for 2 hours. The wash step was repeated. ELISA was developed by addition of TMB substrate. The reaction was incubated at room temperature for 15 minutes and stopped by addition of Red Stop reagent (Neogen Corporation). The results were evaluated to determine the concentration at which 50% HCV efficacy (i.e., EC₅₀) was achieved.

### Example 3

### Measurement of RSV Antiviral Activity

RSV antiviral activity was measured against a RSV minigenome-dependent β-galactosidase expression assay (RSV β-Gal). Cis-acting elements are required for the replication and transcription of a number of negative-strand virus genomes. This requirement can be utilized to develop methods for identifying antiviral compounds. Applicants have applied such methods to, for example, RSV to develop a prototype assay for detecting and quantifying negative-strand RNA viruses. See Olivo et al., “Detection and quantitation of human respiratory syncytial virus (RSV) using minigenorne cDNA and a Sindbis virus replicon: a prototype assay for negative-stranded RNA viruses,” Virology 251:198-205 (1998). In addition, synthetic analogs of genomic RNA have been developed. See Collins et al., “Rescue of synthetic analogs of respiratory syncytial virus genomic RNA and effect of truncations and mutations on the expression of a foreign reporter gene,” Proc. Natl. Acad. Sci. USA 88:9663-7 (1991). These analogues of genomic RNA are referred to as “minigenome RNA”. RNA transcribed from minigenome cDNA contains the cis-acting elements necessary for replication and transcription in RSV-infected cells, and contains a reporter gene(s) in place of viral genes. One of the challenges has been the need to constitutively express high levels of the viral minigenome RNA in the cytoplasm of the cell. To address this challenge, Applicants have developed an infection-independent minigenome expression system for screening anti-RSV drugs. Minigenome replication systems are not true replicons because the cis- and trans-acting elements are not contained on the same RNA molecule. They are, however, functionally equivalent and useful for identifying antiviral compounds. This methodology can be applied to any negative-strand RNA virus by combining knowledge of the specific details of the life cycle of the virus with empiricism.

Transfected cells were plated in 96 well tissue culture treated microplates and allowed to settle for 4 hours at 37° C. and 5% CO₂ (v/v). Test compounds were added to appropriate final concentration with DMSO concentration being held constant at 1% in all wells. No compound controls consisted of cells with media plus DMSO at 1%. Background control wells were non-transfected cells in media plus DMSO at 1%. Cells were incubated in the presence of compound for 24 hours at 37° C. and 5% CO₂ (v/v).

Beta-galactosidase activity was detected using Galacto-Star™ β-Galactosidase Assay Kit (Applied Biosystems of Foster City, Calif., USA). Media and compound were removed from cells. Cell Lysates were prepared by adding lysis solution from the Galacto-Star™ kit and incubating at room temperature for 30 minutes. Galacto-Star™ detection reagent was added and samples were put on an orbital shaker for 1 hour. Beta-galactosidase dependent luminescence was detected with a FLUOstar reader (BMG Labtechnology, Durham, N.C., USA). The results were evaluated to determine the concentration at which 50% RSV efficacy (i.e., ECU) was achieved.

### Example 4

### Measurement of WNV Antiviral Activity

To measure WNV antiviral activity, WN-hRuPac cells were plated in 96-well tissue culture treated microplates and allowed to settle for 4 hr at 37° C. under 5% CO₂ (v/v). Test compounds were added to appropriate final concentration with the DMSO concentration being held constant at 1% in all wells. No-compound controls consisted of cells with media plus DMSO at 1%. Background-control wells consisted of non-transfected cells in media plus DMSO at 1%. Cells were incubated in the presence of the test compound for 24 hr at 37° C. under 5% CO₂ (v/v).

Renilla luciferase activity was detected using the Promega Renilla Luciferase Assay Kit (Promega, Madison, Wis.). The media and compound were removed from the cells. Cell lysates were prepared by adding lysis solution from the Renilla Luciferase Assay kit, and then shaking at room temperature for 15 min. Renilla Luciferase Assay Reagent was added to each well by injection immediately before detection of luminescence. Renilla Luciferase dependent luminescence was detected with a FLUOstar reader (BMG Labtechnology, Durham, N.C.). The results were evaluated to determine the concentration at which 50% WNV efficacy (i.e., EC₅₀) was achieved.

### Example 5

### Measurement of YFV Antiviral Activity

To measure YFV antiviral activity, YF-hRuPac cells were plated in 96-well tissue culture treated microplates and allowed to settle for 4 hr at 37° C. under 5% CO₂ (v/v). Test compounds were added to appropriate final concentration with the DMSO concentration being held constant at 1% in all wells. No-compound controls consisted of cells with media plus DMSO at 1%. Background-control wells were non-transfected cells in media plus DMSO at 1%. The cells were incubated in the presence of the compound for 24 hr at 37° C. under 5% CO₂ (v/v).

Renilla luciferase activity was detected using the Promega Renilla Luciferase Assay Kit (Promega, Madison, Wis.). The media and compound were removed from the cells. Cell lysates were prepared by adding lysis solution from the Renilla Luciferase Assay kit and shaking at room temperature for 15 min. Renilla Luciferase Assay Reagent was added to each well by injection immediately before detection of luminescence. Renilla Luciferase dependent luminescence was detected with a FLUOstar reader (BMG Labtechnology, Durham, N.C.). The results were evaluated to determine the concentration at which 50% YFV efficacy (i.e., EC50) was achieved.

### Example 6

### Measurement of DNG Antiviral Activity

To measure DNG antiviral activity, D2-hRuPac cells were plated in 96-well tissue culture treated microplates and allowed to settle for 4 hr at 37° C. under 5% CO₂ (v/v). Test compounds were added to appropriate final concentration with the DMSO concentration being held constant at 1% in all wells. No-compound controls consisted of cells with media plus DMSO at 1%. Background-control wells were non-transfected cells in media plus DMSO at 1%. The cells were incubated in the presence of the compound for 24 hr at 37° C. under 5% CO₂ (v/v).

Renilla luciferase activity was detected using the Promega Renilla Luciferase Assay Kit (Promega, Madison, Wis.). The media and compound were removed from the cells. Cell lysates were prepared by adding lysis solution from the Renilla Luciferase Assay kit and shaking at room temperature for 15 min. Renilla Luciferase Assay Reagent was added to each well by injection immediately before detection of luminescence. Renilla Luciferase dependent luminescence was detected with a FLUOstar reader (BMG Labtechnology, Durham, N.C.). The results were evaluated to determine the concentration at which 50% DNGV efficacy (i.e., EC₅₀) was achieved.

### Example 7

### Measurement of EBOV Antiviral Activity

EBOV antiviral activity was measured against a EBOV minigenome-dependent Renilla luciferase expression assay (EBOV Rluc). Cis-acting elements are required for the replication and transcription of a number of negative-strand virus genomes. This requirement can be utilized to develop methods for identifying antiviral compounds. Applicants have applied such methods to, for example, EBOV to develop a prototype assay for detecting and quantifying negative-strand RNA viruses. In addition, synthetic analogs of genomic RNA have been developed. These analogues of genomic RNA are referred to as “minigenome RNA”. RNA transcribed from minigenome cDNA contains the cis-acting elements necessary for replication and transcription in EBOV-infected cells, and contains a reporter gene(s) in place of viral genes. One of the challenges has been the need to constitutively express high levels of the viral minigenome RNA in the cytoplasm of the cell. To address this challenge, Applicants have developed an infection-independent minigenome expression system for screening anti-EBOV drugs. Minigenome replication systems are not true replicons because the cis- and trans-acting elements are not contained on the same RNA molecule. They are, however, functionally equivalent and useful for identifying antiviral compounds. This methodology can be applied to any negative-strand RNA virus by combining knowledge of the specific details of the life cycle of the virus with empiricism.

Transfected cells were plated in 96 well tissue culture treated microplates and allowed to settle for 4 hours at 37° C. and 5% CO₂ (v/v). Test compounds were added to appropriate final concentration with DMSO concentration being held constant at 1% in all wells. No compound controls consisted of cells with media plus DMSO at 1%. Background control wells were non-transfected cells in media plus DMSO at 1%. Cells were incubated in the presence of compound for 24 hours at 37° C. and 5% CO₂ (v/v).

Renilla luciferase activity was detected using the Promega Renilla Luciferase Assay Kit (Promega, Madison, Wis.). The media and compound were removed from the cells. Cell lysates were prepared by adding lysis solution from the Renilla Luciferase Assay kit and shaking at room temperature for 15 min. Renilla Luciferase Assay Reagent was added to each well by injection immediately before detection of luminescence. Renilla Luciferase dependent luminescence was detected with a FLUOstar reader (BMG Labtechnology, Durham, N.C.). The results were evaluated to determine the concentration at which 50% EBOV efficacy (i.e., EC₅₀) was achieved.

### Example 8

### Measurement of SINV Antiviral Activity

To measure SINV antiviral activity, SR19-F-luc cells were plated in 96-well tissue culture treated microplates and allowed to settle for 4 hr at 37° C. under 5% CO₂ (v/v). Test compounds were added to appropriate final concentration with the DMSO concentration being held constant at 1% in all wells. No-compound controls consisted of cells with media plus DMSO at 1%. Background-control wells were non-transfected cells in media plus DMSO at 1%. The cells were incubated in the presence of the compound for 24 hr at 37° C. under 5% CO₂ (v/v). Firefly luciferase activity was detected using the Promega Luciferase Assay Kit (Promega, Madison, Wis.). The media and compound were removed from the cells. Cell lysates were prepared by adding lysis solution from the Firefly Luciferase Assay kit and shaking at room temperature for 15 min. Luciferase Assay Reagent was added to each well by injection immediately before detection of luminescence. Luciferase dependent luminescence was detected with a FLUOstar reader (BMG Labtechnology, Durham, N.C.). The results were evaluated to determine the concentration at which 50% SINV efficacy (i.e., EC₅₀) was achieved.

### Example 9

### Measurement of Cell Toxicity

In addition to antiviral activity, the animal cell toxicity of each candidate compound was evaluated. Cellular toxicity of the antiviral compounds was measured in an ATP assay wherein the number of viable cells in a culture was determined by the quanutation of intracellular ATP which denotes the presence of metabolically active cells.

Cells were plated in 96 well microplates treated with tissue culture and allowed to settle for 4 hours at 37° C. and 5% CO₂ (v/v). Test compounds were added to appropriate final concentration with DMSO concentration being held constant at 1% in all wells. No-compound controls consisted of cells with media plus DMSO at 1%. Background control wells were blank wells. The cells were incubated in the presence of compound for 24 hours at 37° C. and 5% CO₂ (v/v).

ATP content of intact cells was measured using the CellTiter-Glo® Assay Kit (Promega Corporation, Madison, Wis., USA). Media and compound were removed from cells. Fresh media and assay reagent were added in equal volumes. The cells were lysed by shaking. The signal was allowed to stabilize and luminescence was read in a FLUOstar reader (BMG. Labtechnology). The results were evaluated to determine the concentration at which 50% cell death occurred (ie., CC0).

As a measure of cellular toxicity to antiviral activity, a therapeutic index (“TI”) for each antiviral compound was calculated as the ratio of the CC₅₀ concentration to the EC₅₀ concentration, or CC₅₀/EC₅₀. NT indicates that the antiviral activity was not tested.

TABLE 3
Primary Screening Data

| | | | | WNV | | DNG | | SINV |
|---|---|---|---|---|---|---|---|---|
| No. | APNo. | HCV Inh | RSV Inh | Inh | YFV Inh | Inh | EBOV Inh | Inh |
| 1 | 0008749 | 176.82 | 99.92 | 65.44 | 77.52 | NT | 39.78 | 34.12 |
| 2 | 0008770 | 153.68 | 99.8 | 49.59 | 71.77 | NT | 20.06 | 70.17 |
| 3 | 0008804 | 32.06 | 86.35 | 38.72 | 41.72 | NT | −44.2 | 76.06 |
| 4 | 0008827 | 105.38 | 47.71 | 28.45 | 81.71 | NT | 6.57 | −22.2 |
| 5 | 0008835 | 210.95 | 98.54 | 89.32 | 84.65 | NT | 86.01 | 29.56 |
| 6 | 0008856 | 186.61 | 91.17 | 79.09 | 77.73 | NT | 42.7 | 76.8 |
| 7 | 0008864 | 103.9 | 76.92 | 34.27 | 64.12 | NT | 85.29 | 7.58 |
| 8 | 0008868 | 98.92 | 75.54 | 49.03 | 66.84 | NT | 4.74 | 34.93 |
| 9 | 0008874 | 98.34 | 88.15 | 33.42 | 76.2 | NT | −17.11 | 48.2 |
| 10 | 0008895 | 34.58 | 99.63 | 81.17 | 86.2 | NT | 38.08 | 15.95 |
| 11 | 0008902 | 142.77 | 99.55 | 74.95 | 79.1 | NT | 62.66 | 81.7 |
| 12 | 0008961 | 10.27 | 31 | 47.55 | 31.3 | NT | 49.84 | 69.37 |
| 13 | 0008993 | 27.42 | 92.11 | 97.96 | 89.46 | NT | 58.97 | −12.07 |
| 14 | 0008998 | 81.25 | 48.39 | 51.64 | 54.6 | NT | −19.48 | 56.69 |
| 15 | 0009047 | 45.15 | 72.61 | 55.88 | 37.55 | NT | 49.21 | 71.95 |
| 16 | 0009088 | 119.12 | 88.46 | 66.8 | 75.93 | NT | 7.83 | 79.45 |
| 17 | 0009095 | 12.27 | 50.71 | 40.34 | 54.05 | NT | −41.99 | 72.12 |
| 18 | 0009096 | 36.93 | 88.61 | 63.92 | 61.45 | NT | −29.52 | 54.4 |
| 19 | 0009104 | 50.86 | 82.31 | 80.6 | 93.16 | NT | 0.97 | 11.88 |
| 20 | 0009105 | 14.77 | 61.41 | 38.42 | 51.04 | NT | 20.99 | 75.54 |
| 21 | 0009108 | 159.14 | 99.4 | 62.68 | 63.1 | NT | 46.49 | 69.66 |
| 22 | 0009109 | 55.15 | 75.11 | 48.84 | 29.93 | NT | 71.99 | −22.71 |
| 23 | 0009116 | 13.7 | 92.05 | 70.31 | 86.26 | NT | −1.14 | 22.76 |
| 24 | 0009121 | 43.67 | 94.65 | 73.87 | 67.3 | NT | 35.26 | 64.07 |
| 25 | 0009141 | 159.05 | 99.94 | 100.01 | 99.96 | NT | 99.43 | 99.75 |
| 26 | 0009144 | 148.4 | 99.87 | 62.23 | 77.2 | NT | 38.75 | 53.3 |
| 27 | 0009145 | 112.54 | 92.15 | 59.99 | 70.24 | NT | −4.87 | 70.57 |
| 28 | 0009155 | 155.15 | 93.72 | 75.19 | 74.96 | NT | 19.24 | 81.33 |
| 29 | 0009219 | 35.32 | 96.2 | 105.38 | 98.36 | NT | 86.43 | −33.83 |
| 30 | 0009231 | −18.05 | 80.34 | 93.74 | 92.46 | NT | 70.97 | −9.95 |
| 31 | 0009278 | 7.21 | 51.69 | −8.6 | −19.15 | NT | −7.34 | −27.5 |
| 32 | 0009279 | −11.11 | 48.26 | −2.69 | −16.9 | NT | 31.95 | −17.57 |
| 33 | 0009291 | 62.25 | 59.91 | 14.47 | 16.14 | NT | 0.43 | −18.41 |
| 34 | 0009304 | 78.58 | 77.33 | 60.36 | 58.45 | NT | 53.75 | 33.97 |
| 35 | 0009311 | 67.54 | 89.94 | 104.17 | 98.7 | NT | 75.18 | 44.89 |
| 36 | 0009320 | 134.64 | 100.01 | 93.65 | 94.4 | NT | 15.49 | −58.78 |
| 37 | 0009347 | 72.91 | 58.02 | 87.76 | 94.42 | NT | 15.68 | 10.27 |
| 38 | 0009457 | 88.36 | 98.17 | 36.51 | 70.84 | NT | −36.38 | 43.24 |
| 39 | 0009462 | 22.43 | 91.64 | 102.06 | 96.56 | NT | 64.09 | 71.87 |
| 40 | 0009604 | 96.08 | 99.35 | 65.56 | 79.58 | NT | 26.29 | 69.54 |
| 41 | 0009605 | 117.05 | 99.41 | 100.09 | 96.55 | NT | 95.23 | 97.21 |
| 42 | 0009615 | 96.54 | 99.95 | 99.98 | 100.02 | NT | 99.47 | 99.81 |
| 43 | 0009634 | 95.39 | 99.97 | 99.93 | 99.98 | NT | 98.68 | 99.68 |
| 44 | 0009645 | 94.7 | 98.89 | 99.87 | 99.98 | NT | 99.61 | 95.83 |
| 45 | 0009664 | 119.36 | 99.86 | 98.98 | 99.98 | NT | 96.22 | 95.74 |
| 46 | 0009668 | 14.98 | 94.69 | 102.92 | 97.68 | NT | 89.99 | −16.88 |
| 47 | 0009686 | 149.49 | 96.37 | 72.96 | 77.66 | NT | 49.52 | 61.43 |
| 48 | 0009716 | 150.66 | 78.04 | 58.46 | 62.13 | NT | 29.36 | 70.33 |
| 49 | 0009723 | 112.79 | 90.16 | 66.36 | 66.53 | NT | 69.88 | 72.47 |
| 50 | 0009725 | 137.73 | 85.53 | 82.05 | 92.18 | NT | 42.62 | 65.76 |
| 51 | 0009775 | 55.45 | 75.45 | 48.96 | 41.77 | NT | 33.72 | 51.91 |
| 52 | 0009820 | 76.45 | 99.14 | 88.93 | 90.02 | NT | 76.92 | 10.19 |
| 53 | 0009880 | 150.13 | 99.97 | 91.92 | 79.94 | NT | 75.56 | 88.12 |
| 54 | 0009887 | 101.4 | 87.89 | 84.36 | 91.12 | NT | 38.44 | 47.82 |
| 55 | 0009891 | 21.9 | 81.45 | 94.92 | 94.33 | NT | 72.78 | 29.11 |
| 56 | 0009923 | 127.9 | 99.01 | 95.68 | 98.73 | NT | 89.34 | 95.79 |
| 57 | 0009943 | 136.41 | 97.91 | 85.74 | 95.84 | NT | 70.8 | 87.7 |
| 58 | 0009958 | 137.26 | 99.82 | 91.54 | 96.59 | NT | 80.18 | 92.75 |
| 59 | 0010006 | 139.73 | 99.7 | 83.68 | 84.58 | NT | 92.18 | 93.47 |
| 60 | 0010643 | 71.32 | 38.15 | 68.27 | 79.4 | NT | 57.43 | 45.75 |
| 61 | 0010651 | 80.11 | 58.69 | 96.89 | 97.71 | NT | 40.89 | 37.45 |
| 62 | 0025608 | −5.83 | 99 | 67.72 | 76.6 | NT | 97.37 | 81.29 |
| 63 | 0025612 | 57.19 | 56.43 | 54.96 | 54.18 | NT | 92.05 | 53.29 |
| 64 | 0025650 | 43.35 | 84.89 | 37.21 | 25.84 | NT | 90.07 | 26.12 |
| 65 | 0025653 | 114.03 | 96.61 | 80.57 | 76.28 | NT | 81.82 | 72.16 |
| 66 | 0025755 | 39.61 | 17.56 | 40.81 | 11.4 | NT | 84.14 | 14.93 |
| 67 | 0025765 | 5.39 | 98.31 | 33.5 | 70.66 | NT | 86.56 | 68.05 |
| 68 | 0025775 | 44.72 | 98.86 | 49.73 | 71.75 | NT | 74.49 | 72.76 |
| 69 | 0025784 | 19.81 | 98.3 | 44.58 | 70.06 | NT | 64.37 | 60.92 |
| 70 | 0025793 | 55.64 | 95.81 | 14.64 | 62.6 | NT | 74.7 | 64.52 |
| 71 | 0025796 | 80.12 | 93.29 | 24.72 | 71.22 | NT | 90.09 | 49.06 |
| 72 | 0025839 | −24.76 | 50.34 | 83.19 | 89.97 | NT | 86.05 | −4.36 |
| 73 | 0025846 | 89.48 | 63.06 | 31.46 | 51.82 | NT | 62.14 | 80.79 |
| 74 | 0025863 | 39.44 | 26.59 | 90.46 | 93.49 | NT | 76.06 | 19.42 |
| 75 | 0025888 | 121.75 | 92.66 | 79.16 | 69.13 | NT | 95.12 | 83.78 |
| 76 | 0025975 | 151.51 | 75.9 | 85.87 | 93.1 | NT | 93.88 | 93.48 |
| 77 | 0080058 | NT | NT | NT | NT | NT | 94.37 | NT |
| 78 | 0080086 | NT | NT | NT | NT | NT | 42.34 | NT |
| 79 | 0080087 | NT | NT | NT | NT | NT | 49.83 | NT |

TABLE 4
Secondary Data for HCV, RSV, WNV and YFV

| | | HCV | HCV | | RSV | RSV | | WNV | WNV | | YFV | YFV | |
|---|---|---|---|---|---|---|---|---|---|---|---|---|---|
| No. | APNO | EC50 | CC50 | HCV TI | EC50 | CC50 | RSV TI | EC50 | CC50 | WNV TI | EC50 | CC50 | YFV TI |
| 1 | 0008749 | 6.52 | 3.83 | 0.59 | 1.89 | 47.55 | 25.16 | 5.7 | 47.55 | 8.34 | 6.34 | 47.55 | 7.5 |
| 2 | 0008770 | 8 | 6.12 | 0.77 | 1.3 | 32.93 | 25.33 | 17.8 | 32.93 | 1.85 | 6.65 | 32.93 | 4.95 |
| 3 | 0008804 | NT | 32.3 | NT | 11.3 | 52.33 | 4.63 | 29.9 | 52.33 | 1.75 | 18.4 | 55.33 | 2.84 |
| 4 | 0008827 | 15.6 | 32.47 | 2.08 | 37 | 72.85 | 1.97 | 75 | 72.85 | 0.97 | 4.26 | 72.85 | 17.1 |
| 5 | 0008835 | 1.76 | 9.67 | 5.49 | 2.8 | 28.55 | 10.2 | 7.15 | 28.55 | 3.99 | 11.1 | 28.55 | 2.57 |
| 6 | 0008856 | 14 | 11.7 | 0.84 | 9.86 | 35.2 | 3.57 | 38.7 | 35.2 | 0.91 | 12.9 | 35.2 | 2.73 |
| 7 | 0008864 | 5.87 | 14 | 2.39 | 10 | 75 | 7.5 | 75 | 75 | 1 | 75 | 75 | 1 |
| 8 | 0008868 | 0.13 | 71.6 | 550.77 | 9.7 | 75 | 7.73 | 59.1 | 75 | 1.27 | 25.8 | 75 | 2.91 |
| 9 | 0008874 | 1.77 | 9.83 | 5.55 | 4.1 | 72.97 | 17.8 | 30.6 | 72.97 | 2.38 | 5.4 | 72.97 | 13.51 |
| 10 | 0008895 | NT | 47.65 | NT | 2.51 | 58.45 | 23.29 | 9.79 | 58.45 | 5.97 | 14.4 | 58.45 | 4.06 |
| 11 | 0008902 | 3.37 | 9.67 | 2.87 | 2.9 | 28.75 | 9.91 | 9.79 | 28.75 | 2.94 | 5.01 | 28.75 | 5.74 |
| 12 | 0008961 | NT | 71 | NT | 6.42 | 71.68 | 11.17 | 19.5 | 71.68 | 3.68 | 61.2 | 71.68 | 1.17 |
| 13 | 0008993 | NT | 75 | NT | 2.93 | 35.37 | 12.07 | 4.78 | 35.37 | 7.4 | 8.88 | 35.37 | 3.98 |
| 14 | 0098998 | 1.3 | 75 | 57.69 | 36.9 | 53.73 | 1.46 | 75 | 53.73 | 0.72 | 9.23 | 53.73 | 5.82 |
| 15 | 0009047 | NT | 35.7 | NT | 7.34 | 72.93 | 9.94 | 75 | 72.93 | 0.97 | 46.4 | 72.93 | 1.57 |
| 16 | 0009088 | 9 | 33.5 | 3.72 | 7.1 | 34.13 | 4.81 | 13.1 | 34.13 | 2.61 | 21 | 34.13 | 1.63 |
| 17 | 0009095 | NT | 30.4 | NT | 37.3 | 75 | 2.01 | 75 | 75 | 1 | 63.6 | 75 | 1.18 |
| 18 | 0009096 | NT | NT | NT | 5.57 | 56.37 | 10.12 | 21.3 | 56.37 | 2.65 | 9.07 | 56.37 | 6.21 |
| 19 | 0009104 | NT | NT | NT | 1.77 | 20.9 | 11.81 | 3.98 | 20.9 | 5.25 | 2.62 | 20.9 | 7.98 |
| 20 | 0009105 | NT | 64.2 | NT | 8.52 | 75 | 8.8 | 75 | 75 | 1 | 26.5 | 75 | 2.83 |
| 21 | 0009108 | 8.09 | 8.02 | 0.99 | 4.16 | 44.8 | 10.77 | 9.62 | 44.8 | 4.66 | 6.26 | 44.8 | 7.16 |
| 22 | 0009109 | 18.1 | 13.65 | 0.75 | 3 | 57.25 | 19.08 | 22.4 | 57.25 | 2.56 | 75 | 57.25 | 0.76 |
| 23 | 0009116 | NT | 75 | NT | 2.91 | 57.93 | 19.91 | 3.76 | 57.93 | 15.41 | 6.48 | 57.93 | 8.94 |
| 24 | 0009121 | NT | NT | NT | 4.5 | 57.87 | 12.86 | 25.6 | 57.87 | 2.26 | 75 | 57.87 | 0.77 |
| 25 | 0009141 | 4.85 | 12.7 | 2.62 | 4.73 | 22.95 | 4.85 | 3.18 | 22.95 | 7.22 | 3.67 | 22.95 | 6.25 |
| 26 | 0009144 | 7.57 | 8.35 | 1.1 | 4.1 | 53.11 | 12.95 | 3.71 | 53.11 | 14.32 | 5.98 | 53.11 | 8.88 |
| 27 | 0009145 | 14 | 24.25 | 1.73 | 6 | 50.7 | 8.45 | 2.78 | 50.7 | 18.24 | 11.8 | 50.7 | 4.3 |
| 28 | 0009155 | 7.47 | 6.82 | 0.91 | 4.58 | 43.8 | 9.56 | 17.4 | 43.8 | 2.52 | 6.79 | 43.8 | 6.45 |
| 29 | 0009219 | NT | 75 | NT | 0.29 | 22.1 | 76.21 | 3.36 | 22.1 | 6.58 | 3.7 | 22.1 | 5.97 |
| 30 | 0009231 | NT | 75 | NT | 1.45 | 55.37 | 38.18 | 4.6 | 55.37 | 12.04 | 5.5 | 55.37 | 10.07 |
| 31 | 0009278 | NT | 75 | NT | 1.55 | 75 | 48.39 | 75 | 75 | 1 | 75 | 75 | 1 |
| 32 | 0009279 | NT | 75 | NT | 3.5 | 75 | 21.43 | 75 | 75 | 1 | 75 | 75 | 1 |
| 33 | 0009291 | 4.06 | 75 | 18.47 | 44.7 | 75 | 1.68 | 38.8 | 75 | 1.93 | 17.8 | 75 | 4.21 |
| 34 | 0009304 | 11.4 | 35.6 | 3.12 | 5.08 | 61.93 | 12.19 | 39.1 | 61.93 | 1.58 | 23 | 61.93 | 2.69 |
| 35 | 0009311 | 8.35 | 52.95 | 6.34 | 1.04 | 37.2 | 35.77 | 2.52 | 37.2 | 14.76 | 3.03 | 37.2 | 12.28 |
| 36 | 0009320 | 6 | 21.3 | 3.55 | 1.46 | 47.3 | 32.4 | 2.38 | 47.3 | 19.87 | 6.78 | 47.3 | 6.98 |
| 37 | 0009347 | 13 | 56 | 4.31 | 1.38 | 41.57 | 30.12 | 4.08 | 41.57 | 10.19 | 2.75 | 41.57 | 15.12 |
| 38 | 0009457 | 9.5 | 19 | 2 | 5.05 | 75 | 14.85 | NT | 75 | NT | 38.7 | 75 | 1.94 |
| 39 | 0009462 | NT | 75 | NT | 1.47 | 26.95 | 18.33 | 5.46 | 26.95 | 4.94 | 3.01 | 26.95 | 8.95 |
| 40 | 0009604 | 5.55 | 5.53 | 1 | 2.43 | 59.97 | 24.68 | 8.39 | 59.97 | 7.15 | 6.45 | 59.97 | 9.3 |
| 41 | 0009605 | 4 | 3.01 | 0.75 | 2.04 | 28.79 | 14.11 | 7.4 | 28.79 | 3.89 | 3.61 | 28.79 | 7.97 |
| 42 | 0009615 | 1.3 | 1.21 | 0.93 | 1.48 | 25.59 | 17.29 | 1.9 | 25.59 | 13.47 | 1.8 | 25.59 | 14.22 |
| 43 | 0009634 | 1.6 | 2.09 | 1.31 | 2.24 | 26.05 | 11.63 | 5.45 | 26.05 | 4.78 | 1.41 | 26.05 | 18.48 |
| 44 | 0009645 | 2.93 | 5.05 | 1.72 | 3.88 | 27.4 | 7.06 | 4.29 | 27.4 | 6.39 | 2.26 | 27.4 | 12.12 |
| 45 | 0009664 | 4.4 | 4.66 | 1.06 | 2.95 | 25.33 | 8.59 | 3.17 | 25.33 | 7.99 | 2.12 | 25.33 | 11.95 |
| 46 | 0009668 | NT | NT | NT | 1.31 | 49.1 | 37.48 | 2.96 | 49.1 | 16.59 | 3.54 | 49.1 | 13.87 |
| 47 | 0009686 | 11 | 7.84 | 0.71 | 3.96 | 56 | 14.14 | 8.66 | 56 | 6.47 | 19.6 | 56 | 2.86 |
| 48 | 0009716 | 10 | 14.1 | 1.41 | 8.32 | 25.6 | 3.08 | 21.3 | 25.6 | 1.2 | 15.9 | 25.6 | 1.61 |
| 49 | 0009723 | 11.1 | 16.3 | 1.47 | 4.39 | 39.27 | 8.94 | 5.58 | 39.27 | 7.04 | 11 | 39.27 | 3.57 |
| 50 | 0009725 | 8.62 | 7.32 | 0.85 | 6.76 | 66.2 | 9.79 | 6.3 | 66.2 | 10.51 | 3.41 | 66.2 | 19.41 |
| 51 | 0009775 | 11.3 | 21.4 | 1.89 | 3.72 | 56.05 | 15.07 | 16 | 56.05 | 3.5 | 15.5 | 56.05 | 3.62 |
| 52 | 0009820 | 12.5 | 70.2 | 5.62 | 2.51 | 44.1 | 17.57 | 10.8 | 44.1 | 4.08 | 4.99 | 44.1 | 8.84 |
| 53 | 0009880 | 4.78 | 8.3 | 1.74 | 3.78 | 49.15 | 13 | 20.95 | 49.15 | 2.35 | 6.47 | 49.15 | 7.6 |
| 54 | 0009887 | 26 | 23.95 | 0.92 | 5.8 | 50.2 | 8.66 | 6.91 | 50.2 | 7.26 | 4.14 | 50.2 | 12.13 |
| 55 | 0009891 | NT | 75 | NT | 5.46 | 48.3 | 8.85 | 13 | 48.3 | 3.72 | 6.07 | 48.3 | 7.96 |
| 56 | 0009923 | 4.56 | 6.69 | 1.47 | 3.33 | 27.84 | 8.36 | 3.5 | 27.84 | 7.95 | 3.51 | 27.84 | 7.93 |
| 57 | 0009943 | 4.98 | 4.23 | 0.85 | 4.56 | 31.03 | 6.8 | 3.6 | 31.03 | 8.62 | 2.76 | 31.03 | 11.24 |
| 58 | 0009958 | 4.95 | 11.8 | 2.38 | 2.34 | 18.67 | 7.98 | 3.8 | 18.67 | 4.91 | 2.65 | 18.67 | 7.04 |
| 59 | 0010006 | 6.07 | 7.45 | 1.23 | 3.26 | 21.2 | 6.5 | 10.9 | 21.2 | 1.94 | 4.4 | 21.2 | 4.82 |
| 60 | 0010643 | 1.4 | 45.5 | 32.5 | 75 | 75 | 1 | 14.6 | 75 | 5.14 | 34.6 | 75 | 2.17 |
| 61 | 0010651 | 0.75 | 46.45 | 61.93 | 32.1 | 45.9 | 1.43 | 22.3 | 45.9 | 2.06 | 10.1 | 45.9 | 4.54 |
| 62 | 0025608 | NT | NT | NT | 3.73 | 43.5 | 11.66 | NT | 43.5 | NT | NT | 43.5 | NT |
| 63 | 0025612 | NT | NT | NT | 72.2 | 70.12 | 0.97 | 44.1 | 70.12 | 1.59 | 51.8 | 70.12 | 1.35 |
| 64 | 0025650 | NT | NT | NT | 22 | 60.52 | 2.75 | 8.9 | 60.52 | 6.8 | 45.9 | 60.52 | 1.32 |
| 65 | 0025653 | 8.48 | 13 | 1.53 | 1.95 | 75 | 38.46 | 5.72 | 75 | 13.11 | 75 | 75 | 1 |
| 66 | 0025755 | NT | NT | NT | 44.8 | 64.62 | 1.44 | 22.9 | 64.62 | 2.82 | 75 | 64.62 | 0.86 |
| 67 | 0025765 | NT | NT | NT | 1.2 | 29.85 | 24.88 | NT | 29.85 | NT | NT | 29.85 | NT |
| 68 | 0025775 | NT | NT | NT | 3 | 51 | 17 | NT | 51 | NT | NT | 51 | NT |
| 69 | 0025784 | NT | 75 | NT | 2.21 | 34.63 | 15.67 | 13.7 | 34.63 | 2.53 | 25.7 | 34.63 | 1.35 |
| 70 | 0025793 | NT | 75 | NT | 3.02 | 45.75 | 15.15 | NT | 45.75 | NT | NT | 45.75 | NT |
| 71 | 0025796 | 8.95 | 39.6 | 4.42 | 4.47 | 49.3 | 11.03 | 13.9 | 49.3 | 3.55 | 58.5 | 49.3 | 0.84 |
| 72 | 0025839 | NT | 75 | NT | 41.7 | 75 | 1.8 | 13.3 | 75 | 5.64 | 20.9 | 75 | 3.59 |
| 73 | 0025846 | 2.32 | 35.2 | 15.17 | 28.95 | 72.29 | 2.5 | 25.8 | 72.29 | 2.8 | 40.3 | 72.29 | 1.79 |
| 74 | 0025863 | NT | NT | NT | 75 | 75 | 1 | 9.72 | 75 | 7.72 | 9.68 | 75 | 7.75 |
| 75 | 0025888 | 0.75 | 10.8 | 14.4 | 5.79 | 23.4 | 4.04 | 9 | 23.4 | 2.6 | 7.48 | 23.4 | 3.13 |
| 76 | 0025975 | 11.3 | 21.5 | 1.9 | 18.2 | 35.5 | 1.95 | 50.4 | 35.5 | 0.7 | 8.92 | 35.5 | 3.98 |
| 77 | 0080058 | NT | NT | NT | 4.75 | 52.6 | 11.07 | NT | 52.6 | NT | NT | 52.6 | NT |
| 78 | 0080086 | NT | 75 | NT | 1.38 | 51.1 | 37.03 | 75 | 51.1 | 0.68 | 17.3 | 51.1 | 2.95 |
| 79 | 0080087 | NT | 75 | NT | 2.2 | 51.25 | 23.3 | 19 | 51.25 | 2.7 | 3.1 | 51.25 | 16.53 |

TABLE 5
Secondary Data for DNG, EBOV and SINV

| No. | APNo. | DNG EC50 | DNG CC50 | DNG TI | EBOV EC50 | EBOV CC50 | EBOV TI | SINV EC50 | SINV CC50 | SINV TI |
|---|---|---|---|---|---|---|---|---|---|---|
| 1 | 8749 | 7.17 | 47.55 | 6.63 | 35.8 | 47.55 | 1.33 | 28.1 | 47.55 | 1.69 |
| 2 | 8770 | 21.3 | 32.93 | 1.55 | 38.7 | 32.93 | 0.85 | 19.3 | 32.93 | 1.71 |
| 3 | 8804 | 34.25 | 52.33 | 1.53 | 65.2 | 52.33 | 0.8 | 1.29 | 52.33 | 40.56 |
| 4 | 8827 | 32.7 | 72.85 | 2.23 | 59.3 | 72.85 | 1.23 | 75 | 72.85 | 0.97 |
| 5 | 8835 | 3.64 | 28.55 | 7.84 | 15.3 | 28.55 | 1.87 | 7.02 | 28.55 | 4.07 |
| 6 | 8856 | NT | 35.2 | NT | NT | 35.2 | NT | 2.3 | 35.2 | 15.3 |
| 7 | 8864 | 17 | 75 | 4.41 | 10.8 | 75 | 6.94 | 75 | 75 | 1 |
| 8 | 8868 | 61.5 | 75 | 1.22 | 54.5 | 75 | 1.38 | 24.2 | 75 | 3.1 |
| 9 | 8874 | 75 | 72.97 | 0.97 | 61.5 | 72.97 | 1.19 | 75 | 72.97 | 0.97 |
| 10 | 8895 | 24.65 | 58.45 | 2.37 | 43.2 | 58.45 | 1.35 | 69.9 | 58.45 | 0.84 |
| 11 | 8902 | NT | 28.75 | NT | 25.9 | 28.75 | 1.11 | 2.8 | 28.75 | 10.27 |
| 12 | 8961 | 69.15 | 71.68 | 1.04 | 45.4 | 71.68 | 1.58 | 6.89 | 71.68 | 10.4 |
| 13 | 8993 | 10.5 | 35.37 | 3.37 | 6.03 | 35.37 | 5.87 | 44.5 | 35.37 | 0.79 |
| 14 | 8998 | 54.3 | 53.73 | 0.99 | 65.6 | 53.73 | 0.82 | 17.6 | 53.73 | 3.05 |
| 15 | 9047 | 71.9 | 72.93 | 1.01 | 71.1 | 72.93 | 1.03 | 5.58 | 72.93 | 13.07 |
| 16 | 9088 | 9.44 | 34.13 | 3.62 | 29.9 | 34.13 | 1.14 | 1.52 | 34.13 | 22.46 |
| 17 | 9095 | 75 | 75 | 1 | 75 | 75 | 1 | 3.83 | 75 | 19.58 |
| 18 | 9096 | 31.5 | 56.37 | 1.79 | NT | 56.37 | NT | 15.3 | 56.37 | 3.68 |
| 19 | 9104 | NT | 20.9 | NT | 75 | 20.9 | 0.28 | NT | 20.9 | NT |
| 20 | 9105 | 42 | 75 | 1.79 | 50.1 | 75 | 1.5 | 4.28 | 75 | 17.52 |
| 21 | 9108 | 20.2 | 44.8 | 2.22 | 47.9 | 44.8 | 0.94 | 12.1 | 44.8 | 3.7 |
| 22 | 9109 | 35.55 | 57.25 | 1.61 | 5.31 | 57.25 | 10.78 | 72.3 | 57.25 | 0.79 |
| 23 | 9116 | 25.8 | 57.93 | 2.25 | 75 | 57.93 | 0.77 | 75 | 57.93 | 0.77 |
| 24 | 9121 | 27.7 | 57.87 | 2.09 | 55.8 | 57.87 | 1.04 | 16.6 | 57.87 | 3.49 |
| 25 | 9141 | 3.89 | 22.95 | 5.9 | 75 | 22.95 | 0.31 | 2.13 | 22.95 | 10.77 |
| 26 | 9144 | 48.2 | 53.11 | 1.1 | 75 | 53.11 | 0.71 | 18.5 | 53.11 | 2.87 |
| 27 | 9145 | 38.4 | 50.7 | 1.32 | 63.4 | 50.7 | 0.8 | 1.88 | 50.7 | 26.97 |
| 28 | 9155 | NT | 43.8 | NT | NT | 43.8 | NT | 2.73 | 43.8 | 16.04 |
| 29 | 9219 | 7.43 | 22.1 | 2.97 | 8.75 | 22.1 | 2.53 | 43 | 22.1 | 0.51 |
| 30 | 9231 | 12 | 55.37 | 4.61 | 12.6 | 55.37 | 4.39 | 75 | 55.37 | 0.74 |
| 31 | 9278 | 75 | 75 | 1 | 75 | 75 | 1 | 75 | 75 | 1 |
| 32 | 9279 | 75 | 75 | 1 | 75 | 75 | 1 | 75 | 75 | 1 |
| 33 | 9291 | 62.75 | 75 | 1.2 | 75 | 75 | 1 | 75 | 75 | 1 |
| 34 | 9304 | 75 | 61.93 | 0.83 | 75 | 61.93 | 0.83 | 75 | 61.93 | 0.83 |
| 35 | 9311 | 11.1 | 37.2 | 3.35 | 6.64 | 37.2 | 5.6 | 75 | 37.2 | 0.5 |
| 36 | 9320 | 16.5 | 47.3 | 2.87 | 75 | 47.3 | 0.63 | 75 | 47.3 | 0.63 |
| 37 | 9347 | 25.5 | 41.57 | 1.63 | 75 | 41.57 | 0.55 | 75 | 41.57 | 0.55 |
| 38 | 9457 | 75 | 75 | 1 | 75 | 75 | 1 | 75 | 75 | 1 |
| 39 | 9462 | 7.1 | 26.95 | 3.8 | 7.22 | 26.95 | 3.73 | 75 | 26.95 | 0.36 |
| 40 | 9604 | 40.6 | 59.97 | 1.48 | 75 | 59.97 | 0.8 | 14.4 | 59.97 | 4.16 |
| 41 | 9605 | 7.91 | 28.79 | 3.64 | 75 | 28.79 | 0.38 | 2.96 | 28.79 | 9.72 |
| 42 | 9615 | 5.05 | 25.59 | 5.07 | 65.8 | 25.59 | 0.39 | 2.99 | 25.59 | 8.56 |
| 43 | 9634 | 1.65 | 26.05 | 15.79 | 75 | 26.05 | 0.35 | 4.08 | 26.05 | 6.38 |
| 44 | 9645 | 3.39 | 27.4 | 8.08 | 75 | 27.4 | 0.37 | 3.57 | 27.4 | 7.67 |
| 45 | 9664 | 5.04 | 25.33 | 5.03 | 75 | 25.33 | 0.34 | 3.23 | 25.33 | 7.84 |
| 46 | 9668 | 10.2 | 49.1 | 4.81 | 7.92 | 49.1 | 6.2 | 49.7 | 49.1 | 0.99 |
| 47 | 9686 | 39.4 | 56 | 1.42 | 75 | 56 | 0.75 | 7.23 | 56 | 7.75 |
| 48 | 9716 | 10.5 | 25.6 | 2.44 | 26.4 | 25.6 | 0.97 | 2.47 | 25.6 | 10.36 |
| 49 | 9723 | 13.2 | 39.27 | 2.97 | 6.88 | 39.27 | 5.71 | 75 | 39.27 | 0.52 |
| 50 | 9725 | NT | 66.2 | NT | NT | 66.2 | NT | NT | 66.2 | NT |
| 51 | 9775 | 60.95 | 56.05 | 0.92 | 52.6 | 56.05 | 1.07 | 13.7 | 56.05 | 4.09 |
| 52 | 9820 | 15.8 | 44.1 | 2.79 | 13.2 | 44.1 | 3.34 | 24.8 | 44.1 | 1.78 |
| 53 | 9880 | 28.7 | 49.15 | 1.71 | 75 | 49.15 | 0.66 | 6.69 | 49.15 | 7.35 |
| 54 | 9887 | 21.9 | 50.2 | 2.29 | 75 | 50.2 | 0.67 | 38.3 | 50.2 | 1.31 |
| 55 | 9891 | 8.53 | 48.3 | 5.66 | 10.4 | 48.3 | 4.64 | 75 | 48.3 | 0.64 |
| 56 | 9923 | 8.48 | 27.84 | 3.28 | 47.5 | 27.84 | 0.59 | 2.33 | 27.84 | 11.95 |
| 57 | 9943 | 6.28 | 31.03 | 4.94 | 75 | 31.03 | 0.41 | 3.97 | 31.03 | 7.81 |
| 58 | 9958 | 6.91 | 18.67 | 2.7 | 4.94 | 18.67 | 3.78 | 4.2 | 18.67 | 4.44 |
| 59 | 0016 | NT | 21.2 | NT | 3.23 | 21.2 | 6.56 | NT | 21.2 | NT |
| 60 | 0010643 | 15.2 | 75 | 4.93 | 75 | 75 | 1 | 12 | 75 | 6.25 |
| 61 | 0010651 | 14.05 | 45.9 | 3.27 | 70.6 | 45.9 | 0.65 | 17.3 | 45.9 | 2.65 |
| 62 | 0025608 | NT | 43.5 | NT | 75 | 43.5 | 0.58 | NT | 43.5 | NT |
| 63 | 0025612 | 44.8 | 70.12 | 1.57 | 9.03 | 70.12 | 7.77 | 75 | 70.12 | 0.93 |
| 64 | 0025650 | 4.19 | 60.52 | 14.44 | 15.5 | 60.52 | 3.9 | 34.1 | 60.52 | 1.77 |
| 65 | 0025653 | 75 | 75 | 1 | 48.8 | 75 | 1.54 | 75 | 75 | 1 |
| 66 | 0025755 | 51.35 | 64.62 | 1.26 | 14.1 | 64.62 | 4.58 | 9.53 | 64.62 | 6.78 |
| 67 | 0025765 | NT | 29.85 | NT | 20.1 | 29.85 | 1.49 | NT | 29.85 | NT |
| 68 | 0025775 | NT | 51 | NT | 68.7 | 51 | 0.74 | NT | 51 | NT |
| 69 | 0025784 | 43.95 | 34.63 | 0.79 | 57.65 | 34.63 | 0.6 | 37.05 | 34.63 | 0.93 |
| 70 | 0025793 | NT | 45.75 | NT | 69.7 | 45.75 | 0.66 | NT | 45.75 | NT |
| 71 | 0025796 | 37.2 | 49.3 | 1.33 | 29.5 | 49.3 | 1.67 | 5.52 | 49.3 | 8.93 |
| 72 | 0025839 | 12.02 | 75 | 6.24 | 17.3 | 75 | 4.34 | 37.1 | 75 | 2.02 |
| 73 | 0025846 | 16.77 | 72.29 | 4.31 | 75 | 72.29 | 0.96 | 5.99 | 72.29 | 12.08 |
| 74 | 0025863 | 4.08 | 75 | 18.38 | 75 | 75 | 1 | 75 | 75 | 1 |
| 75 | 0025888 | 6.09 | 23.4 | 3.84 | 7.15 | 23.4 | 3.27 | 11 | 23.4 | 2.13 |
| 76 | 0025975 | 13.5 | 35.5 | 2.63 | 19.8 | 35.5 | 1.79 | 2.55 | 35.5 | 13.92 |
| 77 | 0080058 | NT | 52.6 | NT | 75 | 52.6 | 0.7 | NT | 52.6 | NT |
| 78 | 0080086 | 57 | 51.1 | 0.9 | 75 | 51.1 | 0.68 | 7.54 | 51.1 | 6.78 |
| 79 | 0080087 | 8.26 | 51.25 | 6.21 | 43.5 | 51.25 | 1.18 | 8.57 | 51.25 | 5.98 |

1. A method for treating a viral infection in an animal, the method comprising administering a therapeutically effective amount of a compound or a pharmaceutically acceptable salt thereof to an animal in need thereof, said compound corresponding to formula (I): 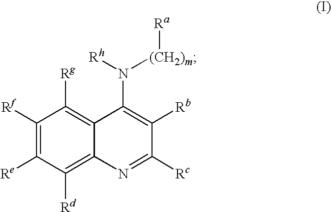
wherein:
m is an integer of 0 to 5; and
as to R^{a}:
R^{a} is selected from the group consisting of hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)(CH₂)₀₋₅R^{X}, —C(O)(CH₂)₀₋₅R^{X}, —OC(O)(CH₂)₀₋₅R^{X}, —C(S)R^{X}, —NHR^{X}, —N═CH—R^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{b}, R^{c}, R^{d}, R^{e}, R^{f}, R^{g} and R^{h}, wherein any member of said strap optionally is substituted; or
R^{a} and R^{b}, R^{c}, R^{d}, R^{e}, R^{f}, R^{g} or R^{h}, together with the atoms to which they are bonded, form an optionally-substituted aryl, heteroaryl, cyclyl or heterocyclyl; and
as to R^{b}:
R^{b} is selected from the group consisting of hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, C(O)R^{X}, C(O)OR^{X}, C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{c}, R^{d}, R^{e}, R^{f}, R^{g} and R^{h}, wherein any member of said strap optionally is substituted; or R^{b} and R^{c}, R^{d}, R^{e}, R^{f}, R^{g} or R^{h}, together with the atoms to which they are bonded, form a moiety selected from carbocyclyl and heterocyclyl, wherein said moiety is optionally substituted with one or more R^{X}; and
as to R^{c}:
R^{c} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{d}, R^{e}, R^{f}, R^{g} and R^{h}, wherein any member of said strap optionally is substituted; or
R^{c} and R^{d}, R^{e}, R^{f}, R^{g} or R^{h}, together with the atoms to which they are bonded, form a moiety selected from carbocyclyl and heterocyclyl, wherein said moiety is optionally substituted with one or more R^{X}; and
as to R^{d}:
R^{d} is selected from the group consisting of hydrogen, halogen, hydroxy, nitro, alkyl, hydroxyalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl optionally substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{e}, R^{f}, R^{g} and R^{h}, wherein any member of said strap optionally is substituted; or
R^{d} and R^{e}, R^{f}, R^{X} or R^{h}, together with the atoms to which they are bonded, form a moiety selected from carbocyclyl and heterocyclyl, wherein said moiety is optionally substituted with one or more R^{X}; and
as to R^{e}:
R^{e} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{f}, R^{g} and R^{h}, wherein any member of said strap optionally is substituted; or
R^{e} and R^{f}, R^{g} or R^{h}, together with the atoms to which they are bonded, form a moiety selected from carbocyclyl and heterocyclyl, wherein said moiety is optionally substituted with one or more R^{X}; and
as to R^{f}:
R^{f} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and: R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{g} and R^{h}, wherein any member of said strap optionally is substituted; or
R^{f} and R^{g} or R^{h}, together with the atoms to which they are bonded, form a moiety selected from carbocyclyl and heterocyclyl, wherein said moiety is optionally substituted with one or more R^{X}; and
as to R^{g}:
R^{g} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{h}, wherein any member of said strap optionally is substituted; or
R^{g} and R^{h}, together with the atoms to which they are bonded, form a moiety selected from carbocyclyl and heterocyclyl, wherein said moiety is optionally substituted with one or more R^{X}; and
as to R^{h}:
R^{h} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{g}, wherein any member of said strap optionally is substituted; and
wherein:
each R^{X} is independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, halogenated aryl, aralkyl, Aralkenyl, C(O)O-aryl, O—CH₂-aryl, S(O)(O)—NH-heteroaryl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyl, and alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl; and
each pair of R^{Y} and R^{Z}, together with the atoms to which they are bonded, form an optionally-substituted moiety selected from carbocyclyl and heterocyclyl, wherein said moiety is optionally substituted with one or more R^{X}.

2. The method according to claim 1, wherein R^{b} and R^{c}, together with the atoms to which they are bonded, form a single ring carbocyclyl, wherein said single ring carbocyclyl is optionally substituted with one or more R^{X}.

3. The method according to claim 2, wherein said single ring carbocyclyl is selected from cycloalkenyl and aryl.

4. The method according to claim 1, wherein R^{d} and R^{e}, together with the atoms to which they are bonded, form a single ring carbocyclyl, wherein said single ring carbocyclyl is optionally substituted with one or more R^{X}.

5. The method according to claim 4, wherein said single ring carbocyclyl is selected from cycloalkenyl and aryl.

6. The method according to claim 1, wherein
m is equal to 0; and
R^{a} is a moiety selected from straight or branched hydroxyalkyl containing 1 to 6 carbon atoms.

7. The method according to claim 1, wherein R^{b}, R^{c}, R^{d}, R^{e}, R^{f}, R^{g} and R^{h} are independently selected from halogen, —OR^{X}, —C(O)OR^{X} and alkyl.

8. The method according to claim 1, wherein:
m is equal to 0; and
R^{a} is a moiety selected from aryl optionally substituted with one or more R^{X} and aryl optionally substituted with R^{Y} and R^{Z}.

9. The method according to claim 1, wherein:
m is equal to 1;
R^{a} is a moiety selected from furyl optionally substituted with one or more R^{X} and furyl optionally substituted with R^{Y} and R^{Z}; and
R^{b} is —OC(O)R^{X}.

10. The method according to claim 1, wherein the compound is selected from the group of compounds, individually represented by the following formula, consisting of: 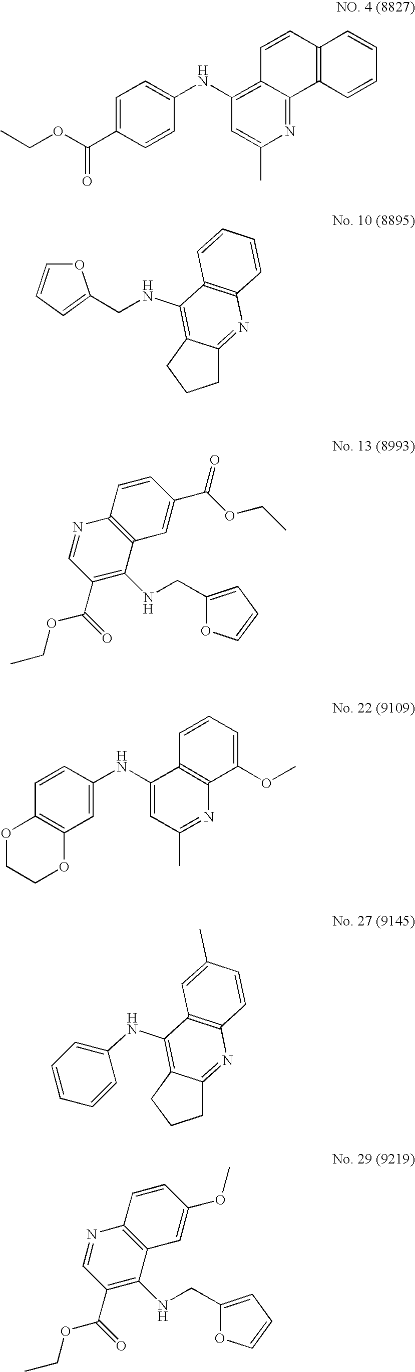 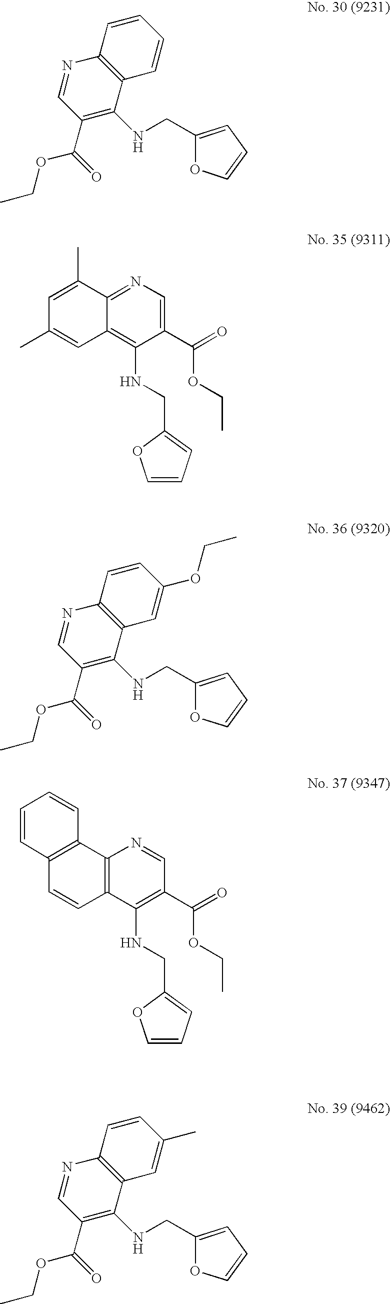 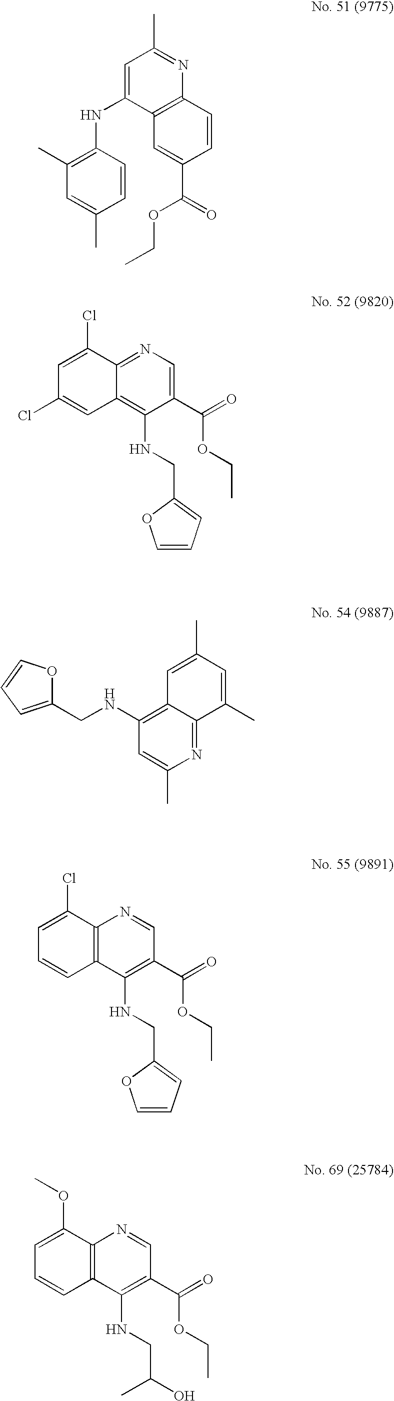
and any combination thereof.

11. A method according to claim 1, wherein the viral infection is caused by an RNA virus.

12. A method according to claim 11, wherein the viral infection is caused by a positive-strand RNA virus.

13. A method according to claim 12, wherein the virus is from a virus family selected from the group consisting of Picornaviridae, Caliciviridae, Astroviridae, Coronaviridae, Togaviridae, and Flaviviridae.

14. A method according to claim 13, wherein the virus is selected from the group consisting of Sindbis virus, rubella virus, hepatitis C virus, West Nile virus, yellow fever virus, tick-borne encephalitis virus, Japanese encephalitis virus, coxsackievirus, enterovirus, hepatitis A virus, severe acute respiratory syndrome virus, astrovirus virus, Dengue fever virus, poliovirus virus, Venezuela encephalitis virus, Western equine encephalomyelitis virus, Eastern equine encephalomyelitis, O'nyong nyong virus, Ross River virus, Chikungunya virus, Rhinovirus, feline calicivirus, murine calicivirus, Norwalk virus, bovine viral diarrhea virus, human coronavirus, Semliki Forest virus, Kunjin virus, Omsk hemorrhagic fever virus, Murray Valley encephalitis virus, Kyasanur Forest disease virus, Rocio virus, and Astrovirus.

15. A method according to claim 13, wherein the virus is hepatitis C virus.

16. A method according to claim 13, wherein the virus is West Nile virus.

17. A method according to claim 13, wherein the virus is yellow fever virus.

18. A method according to claim 11, wherein the infection is caused by a negative-strand RNA virus.

19. A method according to claim 18, wherein the virus is from a virus family selected from the group consisting of Paramyxoviridae, Rhabdoviridae, Filoviridae, Orthomyxoviridae, Bunyaviridae, Bornaviridae, and Arenaviridae.

20. A method according to claim 19, wherein the virus is selected from the group consisting of respiratory syncytial virus, Ebola virus, rabies virus, Lassa fever virus, La Crosse virus, Rift Valley feyer virus, Hantaan virus, California encephalitis virus, influenza virus A, influenza virus B, measles virus, mumps virus, Marburg virus, Bolivian hemorrhagic fever virus, human parainfluenza virus, human metapneumovirus, Nipah virus, Hendra virus, vesicular stomatitis virus, lymphocytic choriomeningitis virus, Junin virus, Bunyamwera virus, Uukuniemi virus, and Crimean-Congo hemorrhagic fever virus.

21. A method according to claim 19, wherein the virus is respiratory syncytial virus.

22. A method according to claim 11, wherein the viral infection is caused by a double-strand RNA virus.

23. A method according to claim 22, wherein the virus is from the Reoviridae virus family.

24. A method according to claim 23, wherein the virus is Colorado tick fever.

25. A method according to claim 1, wherein the viral infection is caused by a DNA virus.

26. A method according to claim 25, wherein the viral infection is caused by a partial-complex DNA virus.

27. A method according to claim 26, wherein the virus is from the Hepadnaviridae virus family.

28. A method according to claim 27, wherein the virus is Hepatitis B virus.

29. A method according to claim 25, wherein the viral infection is caused by a single-strand DNA virus.

30. A method according to claim 29, wherein the virus is from the Paravavoviridae virus family.

31. A method according to claim 30, wherein the virus is human parvovirus.

32. A method according to claim 25, wherein the viral infection is caused by a double-strand DNA virus.

33. A method according to claim 32, wherein the virus is from a virus family selected from the group consisting of Papillomaviridae, Polyomaviridae, and Herpesviridae.

34. A method according to claim 33, wherein the virus is selected from the group consisting of human papillomavirus, JC virus, BK virus, herpes simplex virus 1, herpes simplex virus 2, herpes simplex virus 6, herpes simplex virus 7, herpes simplex virus 8, Eptstein Barr virus, and human cytomegalovirus.

35. A method according to claim 1, wherein the virus is a respiratory virus.

36. A method according to claim 35, wherein the virus is selected from the group consisting of parainfluenza virus, human metapneumovirus, rhinovirus, and hantavirus.

37. A method according to claim 1, wherein the virus is an enteric virus.

38. A method according to claim 37, wherein the virus is selected from the group consisting of enterovirus, rotavirus, and calicivirus.

39. A method according to claim 1, wherein the virus is an encephalitis-causing virus.

40. A method according to claim 39, wherein the virus is selected from the group consisting of West Nile virus and tick-borne encephalitis virus.

41. A method according to claim 1, wherein the virus is a hemorrhagic fever virus.

42. A method according to claim 41, wherein the virus is selected from the group consisting of Ebola virus, Marburg virus, and Lassa fever virus.

43. A method according to claim 1, wherein the method further comprises administering a second antiviral agent to the animal.

44. A method according to claim 43, wherein the second antiviral agent is selected from the group consisting of interferon and ribavirin.

45. A method according to claim 1, wherein the animal is other than human.

46. A method for treating a viral infection in an animal, the method comprising administering a therapeutically effective amount of a compound selected from the group consisting of compounds 1 through 79 of Table 2, a pharmaceutically acceptable salt thereof, and combinations thereof.

47. A pharmaceutical composition for treating a viral infection in an animal:
the composition comprising a therapeutically effective amount of an aminoquinoline compound or a pharmaceutically acceptable salt thereof and a pharmaceutically acceptable carrier wherein the aminoquinoline compound corresponds in structure to formula (I): 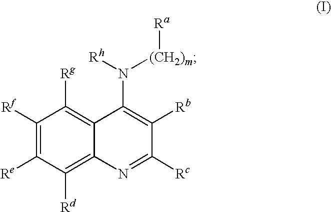
wherein:
m is an integer of 0 to 5; and
as to R^{a}:
R^{a} is selected from the group consisting of hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl., alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)(CH₂)₀₋₅R^{X}, —C(O)(CH₂)₀₋₅OR^{X}, —OC(O)(CH₂)₀₋₅R^{X}, —C(S)R^{X}, —NHR^{X}, —N═CH—R^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{b}, R^{c}, R^{d}, R^{e}, R^{f}, R^{g} and R^{h}, wherein any member of said strap optionally is substituted; or
R^{a} and R^{b}, R^{c}, R^{d}, R^{e}, R^{f}, R^{g} or R^{h}, together with the atoms to which they are bonded, form an optionally-substituted aryl, heteroaryl, cyclyl or heterocyclyl; and
as to R^{b}:
R^{b} is selected from the group consisting of hydrogen, halogen, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, C(O)R^{X}, C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{c}, R^{d}. R^{e}, R^{f}, R^{g} and R^{h}, wherein any member of said strap optionally is substituted; or R^{b} and R^{c}, R^{d}, R^{e}, R^{f}, R^{g} or R^{h}, together with the atoms to which they are bonded, form a moiety selected from carbocyclyl and heterocyclyl, wherein said moiety is optionally substituted with one or more R^{X}; and
as to R^{c}:
R^{c} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{d}, R^{e}, R^{f}, R^{g} and R^{h}, wherein any member of said strap optionally is substituted; or
R^{c} and R^{d}, R^{e}, R^{f}, R^{g} or R^{h}, together with the atoms to which they are bonded, form a moiety selected from carbocyclyl and heterocyclyl, wherein said moiety is optionally substituted with one or more R^{X}; and
as to R^{d}:
R^{d} is selected from the group consisting of hydrogen, halogen, hydroxy, nitro, alkyl, hydroxyalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl optionally substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{e}, R^{f}, R^{g} and R^{h}, wherein any member of said strap optionally is substituted; or
R^{d} and R^{e}, R^{f}, R^{g} or R^{h}, together with the atoms to which they are bonded, form a moiety selected from carbocyclyl and heterocyclyl, wherein said moiety is optionally substituted with one or more R^{X}; and
as to R^{e}:
R^{e} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)ₒ—₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{f}, R^{g} and R^{h}, wherein any member of said strap optionally is substituted; or
R^{e} and R^{f}, R^{g} or R^{h}, together with the atoms to which they are bonded, form a moiety selected from carbocyclyl and heterocyclyl, wherein said moiety is optionally substituted with one or more R^{X}; and
as to R^{f}:
R^{f} is selected from the group consisting of, hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R″, R^{b}, R^{c}, R^{d}, R^{e}, R^{g} and R^{h}, wherein any member of said strap optionally is substituted; or
R^{f} and R^{g} or R^{h}, together with the atoms to which they are bonded, form a moiety selected from carbocyclyl and heterocyclyl, wherein said moiety is optionally substituted with one or more R^{X}; and
as to R^{g}:
R^{g} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{h}, wherein any member of said strap optionally is substituted; or
R^{g} and R^{h}, together with the atoms to which they are bonded, form a moiety selected from carbocyclyl and heterocyclyl, wherein said moiety is optionally substituted with one or more R^{X}; and
as to R^{h}:
R^{h} is selected from the group consisting of hydrogen, halogen, hydroxy, nitro, alkyl, hydroxyalkyl, haloalkyl, alkenyl, alkynyl, cycloalkyl, heterocycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl optionally substituted with one or more R^{X}, aryl optionally substituted with R^{Y} and R^{Z}, heteroaryl optionally substituted with one or more R^{X}, heteroaryl substituted with R^{Y} and R^{Z}, morpholinyl, furanyl, aralkyl, —C(O)R^{X}, —C(O)OR^{X}, —C(S)R^{X}, —NHR^{X}, —OR^{X}, —SR^{X}, —O(PO₂)OR^{X}, —N(R^{X})₂, —(CH₂)₀₋₅(N)(R^{Y})(R^{Z}), —OC(R^{Y})(R^{Z}), —SC(R^{Y})(R^{Z}), —O(PO₂)OC(R^{Y})(R^{Z}), and a strap bonded to a carbon atom of a cyclyl or heterocyclyl comprising any two of R^{a}, R^{b}, R^{c}, R^{d}, R^{e}, R^{f} and R^{g}, wherein any member of said strap optionally is substituted; and
wherein:
each R^{X} is independently selected from the group consisting of hydrogen, halogen, hydroxy, nitro, oxo, alkyl, —O-alkyl, —C(O)-alkyl, —C(O)O-alkyl, —NH(alkyl), —N(alkyl)₂, —NHC(O)-alkyl, haloalkyl, hydroxyalkyl, carboxy, carboxyalkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, aryl, arylakenyl, halogenated aryl, aralkyl, heterocycloalkyl, heteroaryl, heterocycloalkenyl, heterocycloalkylalkyl, heterocycloalkenylalkyl, alkylheterocycloalkyl, alkylheterocycloalkenyl, alkenylheterocycloalkyll, alkenylheterocycloalkenyl, wherein any member of said group optionally is substituted with one or more groups selected from halogen, hydroxy, nitro, oxo, alkyl, —O-alkyl, —C(O)-alkyl, —C(O)O-alkyl, —NH(alkyl), —N(alkyl)₂ and —NHC(O)-alkyl; and each pair of R^{Y} and R^{Z}, together with the atoms to which they are bonded, form an optionally-substituted moiety selected from carbocyclyl and heterocyclyl, wherein said moiety is optionally substituted with one or more R^{X}.

48. A kit comprising a pharmaceutical composition according to claim 47 and, optionally, appropriate diluents, administration devices, and instructions therefore package in a container.
